(19) United States
(12) Patent Application Publication
(10) Pub. No.: US 2006/0217305 A1
(43) Pub. Date: Sep. 28, 2006

(54) Induction of tumor cell senescence by retinoid receptor agonists and antagonists

(75) Inventors: Milos Dokmanovic, New York, NY (US); Yuhong Chen, Albany, NY (US); Igor Roninson, Loudonville, NY (US)

Correspondence Address: Wayne A. Keown, Esq.;Keown & Associates, Suite 1200, 500 West Cummings Park, Woburn, MA, 01801, US

(73) Assignee: Ordway Research Institute, Inc.

(21) Appl. No.: 11/342,253

(22) Filed: Jan. 27, 2006

Related U.S. Application Data
(60) Provisional application No. 60/647,842, filed on Jan. 28, 2005.

(51) Int. Cl. A61K 38/17; A61K 31/203; C12Q 1/68; G01N 33/574; A61K 31/07

(52) U.S. Cl. 514/12; 514/559; 514/725; 435/7.23; 435/6

(57) ABSTRACT

The invention relates to the induction of tumor cell growth arrest. More particularly, the invention relates to the use of retinoic acid receptor (RAR) agonists and antagonists to mediate such growth arrest. The invention provides methods for using RAR-modulating compounds to induce growth arrest, methods for identifying such RAR-modulating compounds, and RAR-modulating compounds identified by such latter methods.

### RELATED APPLICATIONS

This application claims the benefit of U.S. Provisional Application Ser. No. 60/647,842 filed on Jan. 28, 2005, the contents of which are incorporated herein by reference in its entirety.

### BACKGROUND OF THE INVENTION

1. Field of the Invention

The invention relates to the induction of tumor cell growth arrest. More particularly, the invention relates to the use of retinoid receptor agonists and antagonists to mediate such induction of growth arrest.

2. Summary of the Related Art

Retinoids, natural and synthetic derivatives of vitamin A, are used in leukemia treatment and chemoprevention of cancers. These physiological regulators of gene expression were shown to be efficacious in the treatment of promyelocytic leukemia and (to a lesser degree) in chemoprevention of several cancers, in particular breast carcinoma.

Warrell, In Cancer, Principles and Practice of Oncology, V. T. H. S. DeVita and S. A. Rosenbert, eds. (Philadelphia: Lippincot Williams and Wilkins), pp. 489-494 (2001) teaches that retinoid treatment, however, produces a certain amount of systemic toxic responses, such as intracranial hypertension or hyperleukocytosis.

The antitumor effect of retinoids is most often attributed to the induction of differentiation (Altucci and Gronemeyer, Nat. Rev. Cancer 1, 181-193 (2001), but Roninson and Dokmanovic, J. Cell Biochem. 88: 83-94 (2003) teach that these compounds also stop the growth of tumor cells by activating the programs of apoptosis or senescence. Roninson and Dokmanovic, supra also teaches that senescence is observed at the lowest and generally non-toxic concentrations of retinoids, and (ii) it involves upregulation of several growth-inhibitory proteins, including secreted factors that arrest the growth of neighboring non-senescent cells. Senescent tumor cells may therefore be regarded as a reservoir of secreted factors that provide for long-term inhibition of tumor growth.

Dokmanovic et al., PCT/US01/17161 teaches that retinoid-induced senescence of human MCF-7 breast carcinoma cells is associated with increased RNA expression of several intracellular and secreted proteins with known growth-inhibitory activities. These include actin-binding protein EPLIN (Epithelial Protein Lost in Neoplasm) and an ubiquitin-like protein UBD (formerly known as FAT10), as well as secreted proteins insulin-like growth factor-binding protein 3 (IGFBP3) and an extracellular matrix component TGFB1 (formerly known as βIG-h3). Induction of these genes can be used as the test for identifying other compounds that are likely to induce the same form of senescence as retinoids.

Induction of gene expression by retinoids is mediated at the level of transcription, through binding to dimeric transcription factors formed by retinoic acid receptors (RAR) and rexinoid receptors (RXR). The best-known mechanism of action of these retinoid receptors involves their binding to retinoic acid response elements (RARE) in the promoters of retinoid-responsive genes. Nevertheless, Altucci and Gronemeyer, supra teaches that retinoid receptors also affect transcription through RARE-independent mechanisms, such as repression of transcription factor AP-1 (Jun/Fos) and Husmann et al, Biochem. J. 352: 763-772 (2000) teaches that they can act or by modulating the interaction of Sp1 and GC-rich DNA via ternary complex formation.

Remarkably, Dokmanovic et al., Cancer Biology & Therapy 1:24-27 (2002) teaches that only one of 13 genes that were found to be strongly upregulated by retinoids in senescent MCF-7 cells, TRIM31, contains a putative RARE sequence in its promoter, whereas the other genes, including EPLIN, UBD, IGFBP3 and TGFB1, showed no identifiable RARE sequences This suggests that retinoids upregulate these genes via a RARE-independent mechanism, but it is unknown whether this mechanism was mediated by retinoid receptors.

Induction of terminal cell growth arrest is of special interest in anticancer drug development. There is, therefore, a need to develop compounds that can induce growth arrest in tumor cells.

### BRIEF SUMMARY OF THE INVENTION

In a first aspect, the invention provides methods for using one or more retinoic acid receptor (RAR)-modulating compounds to induce growth arrest in proliferating cells and that are efficient in inducing RARE-independent gene expression and that are inefficient in inducing RARE-dependent gene expression.

In a second aspect, the invention provides methods for identifying one or more RAR-modulating compounds that induce growth arrest in proliferating cells and that are efficient in inducing RARE-independent gene expression and that are inefficient in inducing RARE-dependent gene expression with relatively few toxic side effects.

In a third aspect, the invention provides compounds identified by the second aspect of the invention.

### BRIEF DESCRIPTION OF THE DRAWINGS

FIG. 1 shows the effects of RAR agonists (RA and LGD1550, 100 nM each) and RAR antagonist LG100815 (10 μM) on luciferase expression from DR5 RARE-containing promoter in MCF-7 cells. The assays were carried out in triplicate. RA is all-trans retinoic acid. LGD1550 is a pan-RAR agonist. LG100815 is a pan-RAR antagonist.

FIG. 2 shows the effects of retinoid agonists and antagonists on MCF-7 cell growth. The bars represent cell number after 7 days of culture with the addition of DMSO (control), 100 nM RA, 100 nM RAR agonist LGD1550, 100 nM RXR agonist LGD1268, 10 μM RAR antagonist LG100815, and 10 μM RXR antagonist LG101208. Experiments were done in triplicate, and the results are expressed relative to the average of the control.

FIG. 3 shows the effects of retinoid agonists and antagonists on growth and the senescent phenotype of MCF-7 cells. The bars represent percentages of SA-β-gal+ cells after 8 days of treatment with the indicated compounds (in triplicate). The compounds were used at the same concentrations as in FIG. 2.

FIG. 4 depicts microarray analysis of changes in gene expression in MCF-7 cells treated with RAR agonist LGD1550 or RAR antagonist LG100815, plotted using GeneSpring software. The X axis represents different time points of treatment with RAR ligands (0 point correspond to cells cultured for 3 days with DMSO carrier). The Y axis shows changes in gene expression on log scale.

FIG. 5 shows the comparison of changes in gene expression produced by RAR agonist and antagonist. The maximal changes in gene expression for 11,729 probe sets representing genes that show >1.3 fold effect by either the agonist or the antagonist (dots) are plotted on a log scale. Encircled dots correspond to the genes that are affected >5-fold by either ligand. Trend lines (power regression) correspond to the genes with >1.3-fold or >5-fold changes in gene expression The R² values for the regression lines are 0.6955 with n=11729 (for >1.3-fold) and 0.7876 with n=316 (for >5-fold).

FIG. 6 shows the comparison of changes in gene expression produced by RAR agonist and antagonist for the 62 genes listed at the top of Table 3 (similar induction by the agonist and the antagonist; circles) and in Table 4 (preferential induction by the agonist; triangles). The maximal changes in gene expression are plotted on a log scale.

### DETAILED DESCRIPTION OF THE PREFERRED EMBODIMENTS

The invention relates to the induction of tumor cell growth arrest. More particularly, the invention relates to the use of retinoic acid receptor modulators to mediate such induction of growth arrest. The patents and publications cited herein reflect the level of knowledge in the art and are hereby incorporated by reference in their entirety. Any conflict between the teachings of these patents and publications and this specification shall be resolved in favor of the latter.

For purposes of determining the metes and bounds of claims containing this term, the term retinoic acid receptor (RAR) agonist is intended to mean those compounds recognized in the art as those capable of acting through retinoic acid receptors and are efficient at inducing RARE-dependent gene expression. Such compounds include, but are not limited to, all-trans-retinoic acid (RA), 13-cis retinoic acid and LGD1550.

As defined herein, “retinoid-responsive” gene is a gene that is induced by treatment with a known retinoic acid receptor ligand. As defined herein, a retinoic acid receptor ligand is intended to mean a retinoic acid receptor agonist and/or a retinoic acid receptor-modulating compound.

For purposes of determining the metes and bounds of claims containing this term, the term retinoic acid receptor (RAR)-modulating compound is intended to mean those compounds capable of acting through retinoic acid receptors and inducing expression of RARE-independent retinoid-responsive genes but inefficient at inducing RARE-dependent gene expression. Such compounds include, but are not limited to, LG100815.

As defined herein, “RARE-dependent” gene expression refers to the expression of retinoid-responsive genes that are preferentially induced by the RAR agonist relative to RAR-modulating compound (e.g. 3-fold or greater difference in their maximal induction), whether or not such genes contain RARE elements in their promoters.

As defined herein, “RARE-independent retinoid-responsive” gene expression refers to the expression of retinoid-responsive genes that (i) do not contain verified RARE elements in their promoters (see Balmer and Blomhoff, 2005 for a listing of RARE-containing genes) and (ii) are induced by the RAR agonist and the RAR-modulating compound to a similar degree (e.g. no more than 2.5-fold difference in their maximal induction).

In a first aspect, the invention provides methods for using one or more RAR-modulating compounds that are efficient in inducing RARE-independent gene expression and that are inefficient in inducing RARE-dependent gene expression to induce growth arrest in proliferating cells. In preferred embodiments, the proliferating cells are neoplastic cells. In preferred embodiments the proliferating cells are in a mammal, preferable the mammal is a human. In the methods according to this aspect of the invention, preferred compounds include ligands of retinoic acid receptors (RAR). Surprisingly, both agonists and antagonists of RAR induce growth arrest and senescence in proliferating cells, whereas neither agonists nor antagonists of rexinoid receptors (RXR) have this effect.

All-trans retinoic acid (RA) and other RAR agonists induce transcription both through a RARE-dependent mechanism and through RARE-independent mechanisms. The toxicity associated with these compounds may result from the stimulation of RARE-dependent transcription. RAR-modulating antagonists are inefficient in inducing RARE-dependent transcription and therefore should be less toxic. In fact, an RAR antagonist was reported to decrease the toxicity of RAR agonists. (See Standeven et al., Toxicol. Appl. Pharmacol. 138:169-175 (1996)).

In a second aspect, the invention provides methods for identifying one or more RAR-modulating compounds that induce growth arrest in proliferating cells with relatively few toxic side effects that are efficient in inducing RARE-independent gene expression and that are inefficient in inducing RARE-dependent gene expression. In a preferred embodiment, the method according to this aspect of the invention comprises providing proliferating cells, contacting the cells with a test compound, determining the level of RARE-independent retinoid-responsive gene expression (as defined above), determining the level of RARE-dependent gene expression (as defined above), and comparing the ratio of RARE-independent retinoid-responsive gene expression to RARE-dependent gene expression. Test compounds that induce RARE-independent retinoid-responsive gene expression and that have the highest ratio of RARE-independent retinoid-responsive gene expression to RARE-dependent gene expression are determined to be RAR-modulating compounds that induce growth arrest in proliferating cells and should have relatively non-toxic side effects. In preferred embodiments, the level of RARE-independent retinoid-responsive and RARE-dependent gene expression is normalized against cells not treated with the test compound. In certain preferred embodiments, the level of RARE-independent retinoid-responsive and RARE-dependent gene expression is compared to cells treated with a compound known to induce RARE-dependent gene expression, such as RA or another RAR agonist.

In certain embodiments, RARE-independent retinoid-responsive and/or RARE-dependent gene expression can be determined by quantitative reverse-transcription PCR. This embodiment provides a method for identifying one or more RAR-modulating compounds that induce growth arrest in proliferating cells and that are efficient in inducing RARE-independent gene expression and that are inefficient in inducing RARE-dependent gene expression, contacting the cells with a test compound, obtaining cells that have undergone growth arrest, determining the level of expression of RARE-independent retinoid-responsive genes and determining the level of expression of RARE-dependent genes wherein test compounds that increase the expression of RARE-independent retinoid-responsive genes relative to cells not treated with the test compound and that have the highest ratio of the expression of RARE-independent retinoid-responsive genes to the expression of RARE-dependent genes, are determined to be RAR-modulating compounds that are inefficient in inducing RARE-dependent gene expression and induce cell growth arrest.

For example, quantitative reverse-transcription PCR assays in Table 2 show that the ratio of the fold-induction of TGFB1 relative to TRIM31 is 0.19 for 100 nM RA and 0.12 for all three concentrations of RAR agonist LGD1550, but in the case of RAR antagonist LG100815, this ratio increases to 0.56 at 1 μM and 0.38 at 10 μM.

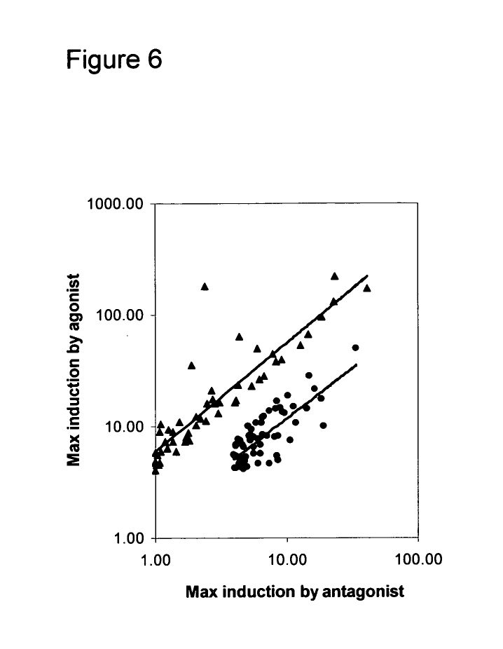

In certain embodiments, RARE-independent retinoid-responsive and/or RARE-dependent gene expression can be determined by hybridization with oligonucleotide or cDNA arrays. For example, FIG. 6 shows that plotting fold increase in gene expression upon treatment with RAR agonist or RAR-modulating antagonist can be used to identify groups of genes that show either similar response to both RAR ligands or preferential response to RAR agonist.

In certain embodiments RARE-independent retinoid-responsive and/or RARE-dependent gene expression can be determined by providing cells transfected with a first gene encoding a first detectable protein operatively linked to a promoter of a RARE-independent retinoid-responsive gene and a second gene encoding a second detectable protein, that is different from and separately detectable in the presence of the first detectable protein, operatively linked to a promoter of a RARE-dependent gene. This embodiment provides a method for identifying one or more RAR-modulating compounds that induce growth arrest in proliferating cells and that are efficient in inducing RARE-independent gene expression and that are inefficient in inducing RARE-dependent gene expression, by contacting the cells with a test compound, measuring the levels of first and second detectable proteins, and comparing the levels of detectable protein expression. Test compounds that induce RARE-independent retinoid-responsive gene expression and that have the highest ratio of RARE-independent retinoid-responsive gene expression to RARE-dependent gene expression are determined to be RAR-modulating compounds that induce growth arrest in proliferating. Preferred detectable proteins include, without limitation, firefly luciferase, Renilla luciferase, beta-galactosidase, chloramphenicol acetyltransferase, horseradish peroxidase, green fluorescent protein, yellow fluorescent protein, cyan fluorescent protein, fluorescent protein DsRed, alkaline phosphatase and immunologically detectable proteins or peptides.

The methods according to this aspect of the invention can be used for testing derivatives of existing RAR agonists or antagonists (see e.g. Hammond et al., Br J. Cancer 85: 453-462 (2001); Standeven et al., Toxicol. Appl. Pharmacol. 138: 169-175 (1996); Toma et al., Int. J. Cancer 78: 86-94 (1998); Yang et al., Breast Cancer Res. Treat. 56: 277-291 (1999)), which can be generated by standard methods of combinatorial chemistry or combinatorial biocatalysis. This method can also be used with other natural or synthetic retinoids with unknown receptor specificity.

In a third aspect, the invention provides compounds identified by the second aspect of the invention, as well as optimized derivatives of such compounds.

The examples below are intended to further illustrate certain preferred embodiments of the invention, and are not intended to limit the scope of the invention.

### EXAMPLE 1

Effect of Compounds on Expression of Genes Associated with Senescence

The following compounds were obtained from Ligand Pharmaceuticals (San Diego, Calif.). LGD1550 is a pan-RAR agonist. LGD1268 is a pan-RXR agonist. LG100815 is a pan-RAR antagonist. LG101208 is a pan-RXR antagonist. The LG100815 compound is a specific RAR antagonist that binds to RAR, but fails to activate its Retinoic Acid Responsive Element (RARE)-dependent transcription transactivation function (see Lee et al, Mol. Cell Biol. 19: 1973-1980 (1999)). Also used was all-trans retinoic acid (RA), the most commonly used RAR agonist. To determine how these compounds affect the expression of retinoid-inducible growth inhibitory genes EPLIN, UBD, IGFBP3 and TGFB1, as well as TRIM31 gene that contains a putative RARE element in its promoter, MCF-7 cells (subline MCF-7 3′SS6) were treated with individual compounds or their combinations for 2 days, and RNA was extracted by standard procedures. In the initial assays, gene expression was analyzed by semi-quantitative reverse transcription-PCR (RT-PCR), as described in Dokmanovic et al., Cancer Biology & Therapy 1:24-27 (2002). In subsequent assays, the initial results were confirmed, expanded and quantitated by real-time RT-PCR (QPCR), using Applied BioSystems 7900HT real-time PCR instrument. β-actin was used as a normalization standard. The primers used for Q-PCR of the corresponding genes are listed in Table 1.

TABLE 1

| | |
|---|---|
| Gene Name | Primer Sequences |

| | | |
|---|---|---|
| IGFBP3 | sense | 5′-TTGCACAAAGACTGCCAAG-3′ |
| | antisense | 5′-AATCAGTTCACCACAAACAG-3′ |
| TRIM31 | sense | 5′-CATGAGGGAACGGAAGCGGG-3′ |
| | antisense | 5′-AACGAGCTTCTTGAGATCGT-3′ |
| UBD | sense | 5′-AATGCTTCCTGCCTCTGTGT-3′ |
| | antisense | 5′-CGCTGTCATATGGGTTGGCA-3′ |
| EPLIN-β | sense | 5′-AGAAGCCGCCCATTCACTGT-3′ |
| | antisense | 5′-CTGATAGGTGGGGACACAGT-3′ |
| TGFBI | sense | 5′-TGCGACTAGCCCCTGTCTAT-3′ |
| | antisense | 5′-GTGGTGCATTCCTCCTGTAG-3′ |
| β-actin | sense | 5′-CTTCCTGGGCATGGAGTC-3′ |
| | antisense | 5′-TGTTGGCGTACAGGTCTTTG-3′ |

cDNA was prepared by reverse transcription with random primer using 4 μg total RNA. 5 μl SYBR Green PCR Master Mix (Applied Biosystems) was mixed with 50 μg cDNA and 0.8 pmol of gene-specific primers and brought up to 10 μl with ultra pure H₂O in 384-well optical plates. For amplification, reaction mixtures were heated for 2 min at 50° C. and 10 min at 95° C., followed by 40 cycles of two-step PCR consisting of 15 sec at 95° C. and 1 min at 60° C., to construct dissociation curves and verify that single PCR products were obtained. PCR products were also analyzed by gel electrophoresis to confirm that a single product of the expected size was amplified. Serial cDNA dilutions were used for primer validation experiments to demonstrate that both target and reference genes had equal amplification efficiency according to the standard curve method. The comparative CT method for relative quantitation of gene expression described by Applied Biosystems was used to determine expression levels for target genes. Experiments were carried out in triplicate for each data point. Sequence Detection Systems software version 2.1 (Applied Biosystems) and Microsoft Excel were used for data analysis.

The results of a representative set of QPCR assays are shown in Table 2.

TABLE 2

| | Fold induction | | | | |
|---|---|---|---|---|---|
| Compounds/Genes | IGFBP3 | EPLIN | UBD | TGFBI | TRIM31 |
| RA (100 nM) | 2.5 ± 0.5 | 3.1 ± 0.7 | 8.5 ± 2.8 | 6.5 ± 1.4 | 34.4 ± 3.9 |
| LGD1550 (100 nM) | 4.2 ± 2.0 | 4.2 ± 0.8 | 9.5 ± 0.2 | 5.1 ± 0.6 | 42.7 ± 1.3 |
| LGD1550 (1 μM) | 3.7 ± 0.7 | 3.2 ± 0.3 | 7.7 ± 1.1 | 3.5 ± 0.4 | 30.3 ± 0.7 |
| LGD1550 (10 μM) | 2.8 ± 1.6 | 4.9 ± 2.5 | 7.3 ± 1.1 | 5.6 ± 0.1 | 45.0 ± 1.4 |
| LG100815 (1 μM) | 2.1 ± 1.1 | 3.8 ± 0.9 | 4.6 ± 0.6 | 4.3 ± 0.7 | 7.7 ± 4.1 |
| LG100815 (10 μM) | 2.0 ± 0.5 | 3.2 ± 1.7 | 2.8 ± 0.6 | 4.4 ± 0.4 | 11.7 ± 4.4 |
| LG100815 (1 μM) + RA | 2.4 ± 1.1 | 1.7 ± 0.5 | 8.0 ± 1.5 | 6.4 ± 2.1 | 30.3 ± 3.3 |
| (100 nM) | | | | | |
| LG100815 (10 μM) + RA (100 nM) | 1.9 ± 0.5 | 0.9 ± 0.2 | 2.2 ± 0.1 | 3.7 ± 0.9 | 10.0 ± 4.6 |
| LGD1268 (100 nM) | 1.2 ± 0.5 | 1.2 ± 0.2 | 1.0 ± 0.1 | 1.9 ± 1.1 | 1.3 ± 0.4 |
| LGD1268 (1 μM) | 0.9 ± 1.1 | 1.0 ± 0.1 | 0.8 ± 0.2 | 2.2 ± 1.0 | 1.4 ± 0.7 |
| LGD1268 (10 μM) | 1.2 ± 0.4 | 1.4 ± 0.2 | 1.0 ± 0.2 | 2.4 ± 0.3 | 3.8 ± 0.7 |
| LG101208 (10 μM) | 1.0 ± 0.2 | 0.8 ± 0.1 | 1.0 ± 0.1 | 0.9 ± 0.2 | 1.0 ± 0.5 |

The results of this analysis show the following. The pan-RAR agonist LGD1550 induced all five genes to approximately the same extent as RA. The pan-RXR agonist LGD1268 induced only one of five genes (TGFB1) but the extent of induction was much lower than the effect of RA or LGD1550. These findings indicate that retinoid-inducible gene expression is activated primarily through RAR.

Whereas the pan-RXR antagonist LG101208 had no effect on gene expression, the pan-RAR antagonist LG100815, surprisingly, induced the expression of all five genes. The magnitude of induction by LG100815 was similar to or slightly lower than that of RA or LGD1550 for EPLIN, IGFBP3, TGFB1 and UBD, but 3-4 fold lower for TRIM31 (the only gene that contains RARE in its promoter). When LG100815 was combined with RA, it decreased the induction of gene expression by RA to the levels that were similar to or (in the case of EPLIN) lower than the levels observed with LG100815 alone, with the biggest decrease from RA-induced levels observed for TRIM31. This finding was consistent with the notion that LG100815, while sharing with RA the ability to induce gene expression, also partially antagonizes the inducing effect of RA.

### EXAMPLE 2

Effect of LG100815 on RARE-Dependent Induction of Transcription

To confirm the ability of LG100815 to antagonize RARE-dependent induction of transcription analysis of firefly luciferase expression from a RARE-containing artificial promoter DR5 (Stratagene, catalog number 240119) was performed. Cells were plated to the density of 3×10⁵ in P60 24 hrs before transient transfection. DR5 reporter plasmid (4 μg) was mixed with the SV40-driven-Renilla luciferase control plasmid (0.04 μg) and transfected using Lipofectamine Plus (Life Technologies/Invitrogen, Carlsbad, Calif.) as described by the manufacturer. 3 hrs after transfection, cells were rinsed three times with PBS, trypsinized and replated in a 12-well plate to the density of 5×10⁴ cells per well. Retinoid agonists and antagonists were added 48 hrs later, and the luciferase assay was performed after another 24 hrs.

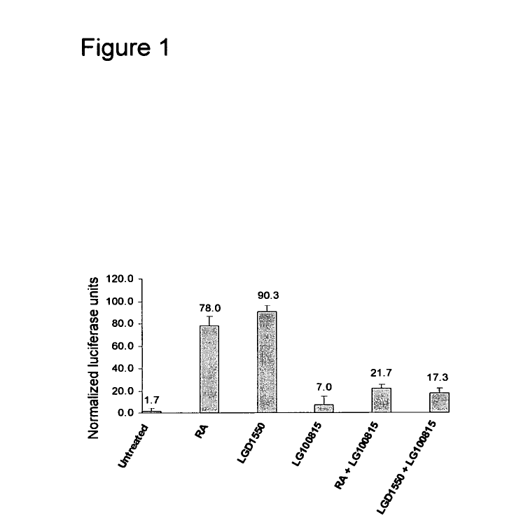

FIG. 1 shows the results of DR5-luciferase transient transfection assays, carried out in the presence of LGD1550, RA, and LG100815, alone or in pairwise combinations. 100 nM concentrations of RA or LGD1550 agonists strongly activated the RARE-containing promoter approximately 50-fold, whereas 10 μM of LG100815 antagonist (the concentration used in the literature for maximal effect) produced an order of magnitude weaker (4.2-fold) induction. On the other hand, the addition of LG100815 to RA or RAR agonist LGD1550 diminished the induction of transcription by the latter compounds 2.5-3 times. These results confirm that LG100815 is inefficient in stimulating RARE-dependent transcription relative to RAR agonists and that it antagonizes the effect of RAR agonists on such transcription.

### EXAMPLE 3

### Induction of Senescence in Treated Cells

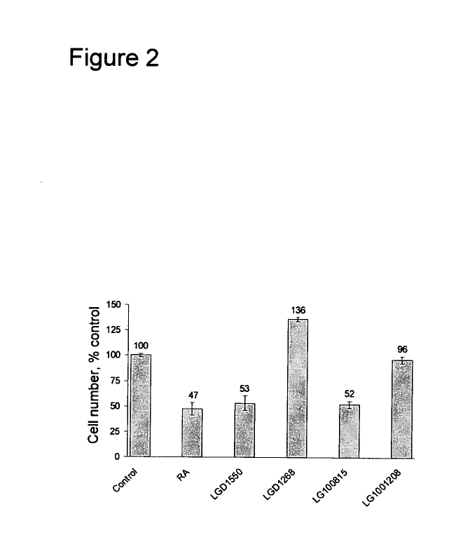

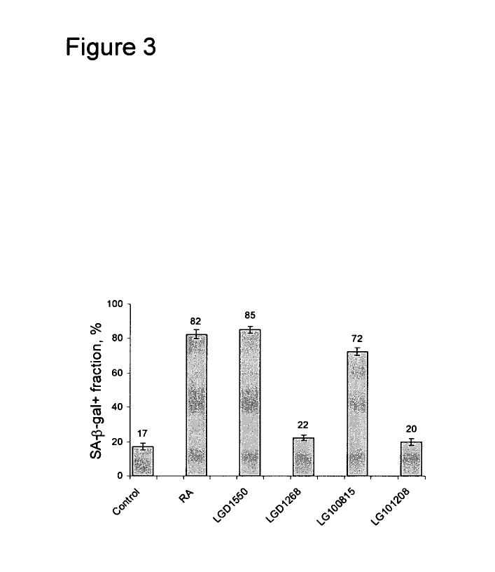

Induction of senescence-associated growth-inhibitory genes by the RAR agonist LGD1550 and the RAR antagonist LG100815 (Table 2) suggests that these compounds may also be able to induce senescence in the treated cells. To test this, we have analyzed the effects of different compounds on the growth of MCF-7 cells, as measured by the cell number after 7 days exposure to the compounds (FIG. 2) and on the fraction of cells expressing the senescence-associated β-galactosidase activity (SA-β-gal), a marker of senescence, after 7-day treatment with the compounds (FIG. 3).

The RAR agonist LGD1550 inhibited the cell growth and induced SA-β-gal to an extent similar to that of RA, demonstrating that RAR stimulation is sufficient to induce senescence. The RXR agonist LGD1268 did not inhibit cell growth and did not induce the senescent phenotype; in fact, LGD1268 treatment produced a modest but reproducible increase in cell growth. The RXR antagonist LG101208 had no effect on the cell growth or the senescent phenotype. The RAR antagonist LG100815 produced both growth inhibition and the induction of the senescence marker; at the highest concentration (10 μM), its effects were similar to those of RA and LGD1550. Hence, an RAR antagonist that inhibits transactivation of RARE-dependent transcription but stimulates the expression of senescence-associated growth-inhibitory genes, induces cell growth arrest and senescence in MCF-7 breast carcinoma cells.

### EXAMPLE 4

RAR Agonist and Antagonist Produce Similar Effects on Global Gene Expression

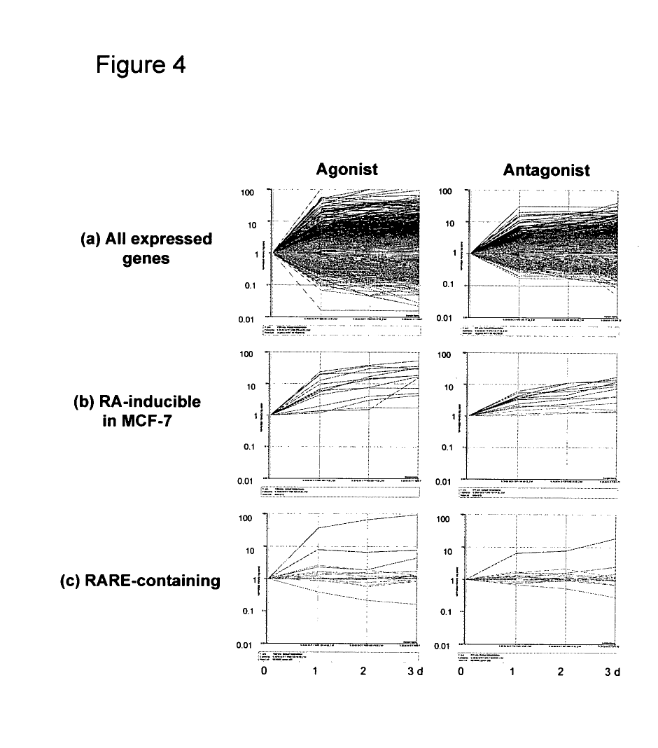

To determine the effects of the RAR agonist and antagonist on the expression of essentially all the human genes, MCF-7 cells were treated with 100 nM of RAR agonist LGD1550 or 10 μM of RAR antagonist LG100815, concentrations that provide maximal induction of gene expression according to Q-PCR assays (as discussed above). Cells were treated for 24, 48 or 72 hrs, and total RNA from the untreated or treated cells was isolated using Qiagen's RNeasy Total RNA Isolation Kit. For gene expression profiling, RNA samples were provided to the microarray service facility of the Wadsworth Center Genomics Institute, which carried out biotinylated target preparation and hybridization with Affymetrix U 133 2.0 Plus oligonucleotide microarrays containing 56,000 probe sets representing 48,500 human transcripts. The hybridization signals were normalized using GCRMA procedure and analyzed using GeneSpring software (Silicone Genetics). The results of the analysis (FIG. 4a) showed good concordance among different time points (e.g. 85-93% of genes induced or inhibited ≧1.5-fold on day 2 were also induced or inhibited ≧1.2-fold, respectively, on day 3). The effects of the agonist and the antagonist also agreed with the results of the earlier analysis of the effects of RA on MCF-7 cells, with all 13 genes previously shown by RT-PCR to be induced in that system (Dokmanovic et al., 2002) also showing induction in the present microarray analysis (FIG. 4b).

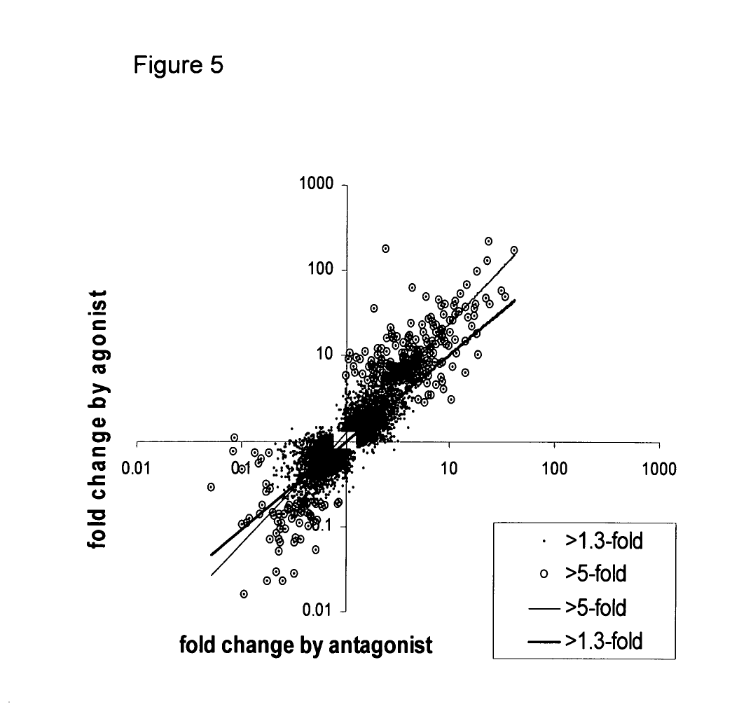

Strikingly, the effects of the RAR agonist and the RAR antagonist were exceedingly similar. 74% of the genes showing ≧1.5-fold induction and 77% of the genes showing ≧1.5-fold inhibition by the antagonist were also induced or inhibited, respectively, at least 1.3-fold by the agonist, and vice versa (77% and 69%, respectively). FIG. 5 plots (on the log scale) the maximal changes in gene expression (at any time point) produced by the agonist versus those produced by the antagonist for 11,729 probe sets that showed >1.3-fold changes in gene expression after treatment with either the agonist or the antagonist. The effects of the agonist and antagonist on gene expression show highly significant correlation. The regression through all the data points has an r squared value of 0.6955 with n=11729 (Student's t test yields p<<0.0001). The regression line has a slope of 1.027+/−0.006 (FIG. 5), indicating that the RAR agonist and the RAR antagonist have the same effect on the majority of the affected genes. The similarity of the overall effects of the agonist and the antagonist on gene expression agrees with the results of QPCR analysis of selected genes (see Table 2 above) but contrasts with an order of magnitude weaker effect of the antagonist on RARE-dependent transcription (see FIG. 1).

On the other hand, 316 genes showing the strongest (>5-fold) induction or inhibition by RAR ligands were significantly more responsive to the agonist than to the antagonist (at p<<0.0001), with the regression slope increasing to 1.297+/−0.038 (this translates to approximately 2-fold stronger average effect of the agonist relative to the antagonist) (FIG. 5). Whether preferential induction of the most responsive genes by the agonist could indicate the presence of RARE sequences in the corresponding promoters was considered. Indeed, the gene showing the strongest induction by the agonist (FIG. 5) encodes RA-metabolizing enzyme CYP26A1 (induced 220-fold by the agonist and 23.5-fold by the antagonist), which was reported to contain two synergistically acting RARE sequences in its promoter (Loudig et al., 2005). The effects of the agonist and the antagonist on the expression of 40 human genes, identified by Balmer and Blomhoff (Balmer and Blomhoff, 2005) as containing canonical and evolutionarily conserved RARE sequences in their promoters was examined. Only seven of these 40 genes were induced ≧1.5-fold in MCF-7 cells by the RAR agonist and just three genes were induced by the antagonist. RARE-containing genes induced by both ligands showed 3-5 fold stronger response to the agonist than to the antagonist (FIG. 4c). In particular, HOXA1 was induced 95-fold by the agonist but only 18-fold by the antagonist, HOXA4 was induced 7.8-fold by the agonist and 2.5-fold by the antagonist, and RBP1 was induced 4.4-fold by the agonist and 1.5-fold by the antagonist. Hence, RARE-containing genes that are responsive to RAR ligands in MCF-7 cells indeed show stronger response to the agonist than to the antagonist. However, when promoter sequences of ten randomly chosen genes that show preferential induction by the agonist for the presence of putative RARE sequences were examined (this analysis was carried out using MatInspector program as previously described (Dokmanovic et al., 2002)), only one of ten promoters was found to contain putative RARE sequences. Therefore the majority of genes showing preferential response to the agonist do not contain RARE sequences in their promoters. Without wishing to be bound to any particular theory, this finding can be explained as follows. The agonist induces a small number of genes that have RARE elements in their promoter, whereas the antagonist is less efficient in inducing such genes. Some of these RARE-containing genes in their turn cause the activation of a number of other genes, which don't contain RARE elements. The latter genes, which are also preferentially induced by the agonist, can therefore also be regarded as RARE-dependent, despite the absence of RARE in their promoters.

RNA or protein products of genes that are induced to a similar level by both the RAR agonist and the RAR antagonist can be used as reporters in screening for compounds with properties similar to LG0100815. RNA or protein products of genes from this group (or promoter constructs for such genes) can be used as reporters in screening for compounds that mimic the effect of retinoids. Table 3, as shown below, lists a set of 508 genes chosen as preferred reporters. These genes were selected by being strongly (at least 2-fold) induced by both the agonist and the antagonist relative to untreated cells and showing no more than two-fold difference between their induction by the agonist and the antagonist. 62 genes listed at the top of Table 3 are particularly preferred reporters, as they are most strongly (at least 4-fold) induced by both the agonist and the antagonist. Table 4, as shown below, lists a set of 53 genes that can be used as preferred markers to discriminate between RARE-dependent and RARE-independent induction of transcription. These genes were chosen by being induced at least 3-fold by the agonist relative to untreated cells and also showing at least 4 times stronger induction by the agonist than by the antagonist. FIG. 6 plots the maximal fold induction of gene expression produced by the agonist versus that produced by the antagonist for the genes listed in Table 3 (particularly preferred reporters only) and in Table 4, with the corresponding trend lines. The relative effects of a tested compound on the genes in Table 3 and Table 4 should indicate whether the compound behaves as a RAR agonist or RAR antagonist. For example, one can select matching pairs of genes from Table 3 and Table 4 that are induced by the agonist to approximately the same degree. A compound that mimics the effects of the agonist should induce such genes to a similar level, whereas a compound that behaves like an antagonist should induce the gene from Table 3 to a much greater degree than the gene from Table 4.

TABLE 3
Genes showing strong induction by both RAR agonist and RAR antagonist.

| | | | Maximal effect of | | Maximal effect of | | Relative |
|---|---|---|---|---|---|---|---|
| | | | agonist | | antagonist | | maximal |
| | | Affymetrix probe | Fold | Raw | Fold | Raw | induction |
| Genbank ID | Gene name | ID | induction | signal | induction | signal | (antag/agon) |

Particularly preferred

| | | | | | | | |
|---|---|---|---|---|---|---|---|
| NM_014059 | RGC32 | 218723_s_at | 49.56 | 458.52 | 33.97 | 313.09 | 0.69 |
| AI693140 | LOC283824 | 213725_x_at | 17.58 | 248.67 | 18.59 | 261.87 | 1.06 |
| NM_000623 | BDKRB2 | 205870_at | 21.40 | 559.77 | 16.36 | 426.27 | 0.76 |
| AF146343 | NR5A2 | 208343_s_at | 28.17 | 391.17 | 14.95 | 207.89 | 0.53 |
| AL353944 | RUNX2 | 232231_at | 14.40 | 349.06 | 14.21 | 343.00 | 0.99 |
| AI935586 | | 235350_at | 15.09 | 349.00 | 11.32 | 260.91 | 0.75 |
| NM_003999 | OSMR | 205729_at | 10.63 | 74.54 | 11.70 | 82.16 | 1.10 |
| AF052094 | EPAS1 | 200878_at | 18.63 | 5620.46 | 10.25 | 3081.12 | 0.55 |
| NM_006329 | FBLN5 | 203088_at | 10.09 | 70.00 | 19.01 | 131.99 | 1.88 |
| NM_024430 | PSTPIP2 | 219938_s_at | 13.08 | 182.67 | 9.79 | 136.87 | 0.75 |
| NM_005564 | LCN2 | 212531_at | 13.27 | 19375.20 | 9.42 | 13703.50 | 0.71 |
| X68285 | GK | 215977_x_at | 14.75 | 152.27 | 9.06 | 93.02 | 0.61 |
| AL359338 | CMYA5 | 233520_s_at | 16.94 | 369.96 | 8.54 | 185.81 | 0.50 |
| NM_000584 | IL8 | 202859_x_at | 14.36 | 139.56 | 8.24 | 80.15 | 0.57 |
| AF228422 | NMES1 | 223484_at | 8.21 | 1084.34 | 8.69 | 1148.89 | 1.06 |
| BE500942 | C6orf155 | 226810_at | 7.95 | 280.01 | 8.18 | 286.84 | 1.03 |
| NM_012449 | STEAP | 205542_at | 7.41 | 109.11 | 10.73 | 157.35 | 1.45 |
| NM_003128 | SPTBN1 | 200672_x_at | 13.66 | 184.68 | 7.37 | 99.28 | 0.54 |
| AW444617 | DCDC2 | 222925_at | 8.21 | 561.12 | 7.16 | 487.38 | 0.87 |
| AI953847 | IBRDC2 | 228153_at | 12.34 | 794.25 | 6.74 | 432.13 | 0.55 |
| NM_016235 | GPRC5B | 203632_s_at | 11.99 | 360.51 | 6.54 | 197.00 | 0.55 |
| NM_006763 | BTG2 | 201236_s_at | 8.31 | 724.17 | 6.54 | 567.74 | 0.79 |
| NM_015577 | RAI14 | 202052_s_at | 10.63 | 91.09 | 6.50 | 55.48 | 0.61 |
| BF939996 | | 231098_at | 6.87 | 52.25 | 6.31 | 47.76 | 0.92 |
| NM_004591 | CCL20 | 205476_at | 7.59 | 152.64 | 6.14 | 123.50 | 0.81 |
| NM_018302 | FLJ11017 | 219450_at | 10.81 | 437.79 | 5.83 | 235.07 | 0.54 |
| AI655057 | RIT1 | 239843_at | 5.71 | 71.05 | 6.32 | 78.33 | 1.11 |
| NM_001415 | EIF2S3 | 205321_at | 6.48 | 1267.17 | 5.68 | 1106.51 | 0.88 |
| AI918054 | | 1555893_at | 5.65 | 46.92 | 5.63 | 46.83 | 1.00 |
| AB028976 | SAMD4 | 212845_at | 7.89 | 119.13 | 5.60 | 84.24 | 0.71 |
| U58515 | CHI3L2 | 213060_s_at | 5.46 | 67.16 | 8.42 | 103.54 | 1.54 |
| NM_003979 | GPCR5A | 203108_at | 9.37 | 2383.55 | 5.40 | 1367.87 | 0.58 |
| NM_024087 | ASB9 | 205673_s_at | 7.53 | 195.71 | 5.35 | 138.28 | 0.71 |
| AI948503 | ABCC4 | 203196_at | 8.41 | 220.20 | 5.33 | 139.04 | 0.63 |
| AL161725 | DOCK8 | 232843_s_at | 8.08 | 243.37 | 5.17 | 155.26 | 0.64 |
| M55580 | SAT | 210592_s_at | 9.95 | 4553.85 | 5.08 | 2313.75 | 0.51 |
| U73778 | COL12A1 | 231766_s_at | 5.00 | 125.94 | 8.63 | 216.58 | 1.73 |
| AB062292 | CTNNB1 | 1554411_at | 5.30 | 101.79 | 4.92 | 94.18 | 0.93 |
| AL031680 | PARD6B | 214827_at | 4.94 | 109.64 | 4.85 | 107.18 | 0.98 |
| W46388 | SOD2 | 215223_s_at | 4.90 | 1590.32 | 4.83 | 1566.99 | 0.98 |
| BE207758 | ARRB1 | 222912_at | 6.51 | 73.19 | 4.76 | 53.36 | 0.73 |
| NM_001970 | EIF5A | 201123_s_at | 4.66 | 2495.21 | 4.68 | 2498.82 | 1.01 |
| NM_005204 | MAP3K8 | 205027_s_at | 4.66 | 224.27 | 6.04 | 289.39 | 1.30 |
| NM_014322 | OPN3 | 219032_x_at | 6.88 | 318.00 | 4.62 | 212.55 | 0.67 |
| AI888150 | | 228494_at | 4.59 | 36.28 | 7.42 | 58.67 | 1.62 |
| AW264102 | FAM43A | 227410_at | 4.59 | 130.51 | 4.57 | 129.32 | 1.00 |
| BF575213 | MGC5618 | 221477_s_at | 5.35 | 727.20 | 4.46 | 605.90 | 0.83 |
| L39833 | KCNAB1 | 210078_s_at | 7.54 | 77.61 | 4.44 | 45.44 | 0.59 |
| NM_006275 | SFRS6 | 206108_s_at | 4.74 | 365.70 | 4.43 | 340.91 | 0.94 |
| AL021977 | MAFF | 36711_at | 4.68 | 146.54 | 4.40 | 137.09 | 0.94 |
| M34421 | PSG9 | 209594_x_at | 4.35 | 53.49 | 4.99 | 61.09 | 1.15 |
| AB046692 | AOX1 | 205082_s_at | 4.36 | 97.94 | 4.34 | 97.10 | 1.00 |
| AA156240 | RAI3 | 212444_at | 7.59 | 752.15 | 4.31 | 427.30 | 0.57 |
| AL035689 | NCOA7 | 225344_at | 5.36 | 1202.13 | 4.24 | 953.11 | 0.79 |
| Z21533 | HHEX | 215933_s_at | 6.67 | 146.87 | 4.16 | 91.23 | 0.62 |
| M63310 | ANXA3 | 209369_at | 6.74 | 1062.27 | 4.15 | 652.48 | 0.62 |
| AA195485 | ZKSCAN1 | 225221_at | 4.24 | 1791.93 | 4.14 | 1742.53 | 0.98 |
| U22178 | MSMB | 210297_s_at | 4.11 | 60.62 | 4.72 | 69.21 | 1.15 |
| NM_001710 | BF | 202357_s_at | 5.43 | 968.70 | 4.11 | 733.54 | 0.76 |
| AW025141 | | 228400_at | 4.25 | 87.08 | 4.04 | 82.34 | 0.95 |
| U49396 | P2RX5 | 210448_s_at | 5.46 | 31.21 | 4.01 | 22.60 | 0.74 |
| AB037925 | NFKBIZ | 223218_s_at | 5.61 | 1705.70 | 4.01 | 1221.80 | 0.72 |

Other preferred

| | | | | | | | |
|---|---|---|---|---|---|---|---|
| AF145712 | NRP1 | 210510_s_at | 6.89 | 363.13 | 3.96 | 208.02 | 0.58 |
| AA826324 | RASEF | 235144_at | 7.35 | 559.95 | 3.93 | 298.12 | 0.53 |
| AA807060 | LOC283357 | 235151_at | 3.90 | 20.80 | 4.21 | 22.47 | 1.08 |
| AW341649 | TP53INP1 | 225912_at | 4.84 | 1086.95 | 3.90 | 872.37 | 0.81 |
| AW501195 | FBXW2 | 241736_at | 4.40 | 32.52 | 3.88 | 28.63 | 0.88 |
| AC004522 | ZNF36 | 214900_at | 4.90 | 297.50 | 3.87 | 234.13 | 0.79 |
| AW052084 | WIPI49 | 213836_s_at | 7.51 | 392.30 | 3.87 | 200.83 | 0.51 |
| AI809961 | PFAAP5 | 221899_at | 3.82 | 52.97 | 3.96 | 54.69 | 1.04 |
| BE502982 | YPEL2 | 227020_at | 3.80 | 368.71 | 4.13 | 398.97 | 1.09 |
| AI129310 | C13orf18 | 44790_s_at | 3.97 | 134.75 | 3.79 | 128.24 | 0.96 |
| AF278532 | NTN4 | 223315_at | 7.44 | 711.60 | 3.77 | 359.24 | 0.51 |
| AL391688 | SYTL4 | 227703_s_at | 3.75 | 140.75 | 3.83 | 142.98 | 1.02 |
| BF576053 | CFL2 | 224663_s_at | 3.74 | 77.99 | 3.78 | 78.46 | 1.01 |
| AI608725 | ICAM1 | 202637_s_at | 5.94 | 198.71 | 3.67 | 122.86 | 0.62 |
| AF345568 | GPR81 | 224131_at | 6.53 | 40.70 | 3.61 | 22.42 | 0.55 |
| AL120021 | LOC339924 | 226158_at | 3.57 | 316.12 | 3.91 | 346.54 | 1.10 |
| AA653300 | ZNF36 | 214670_at | 5.24 | 939.28 | 3.57 | 636.23 | 0.68 |
| AI201594 | | 43511_s_at | 4.78 | 269.49 | 3.57 | 200.51 | 0.75 |
| NM_016548 | GOLPH2 | 217771_at | 6.67 | 62.11 | 3.55 | 32.87 | 0.53 |
| AI569872 | FZD4 | 229441_at | 3.65 | 54.08 | 3.54 | 52.19 | 0.97 |
| L25541 | LAMB3 | 209270_at | 3.54 | 66.53 | 3.49 | 65.60 | 0.99 |
| NM_018191 | RCBTB1 | 218352_at | 6.11 | 197.41 | 3.47 | 111.66 | 0.57 |
| AI810244 | MGC7036 | 227983_at | 4.75 | 384.25 | 3.46 | 278.58 | 0.73 |
| AB032963 | ATP8B2 | 226771_at | 4.89 | 573.51 | 3.43 | 400.38 | 0.70 |
| BC005297 | KMO | 211138_s_at | 3.39 | 55.36 | 6.35 | 103.79 | 1.87 |
| AI271418 | LOC150763 | 228198_s_at | 5.91 | 284.69 | 3.38 | 162.22 | 0.57 |
| BG036668 | C9orf3 | 212848_s_at | 3.36 | 153.82 | 3.51 | 160.13 | 1.04 |
| AA928542 | LOC91137 | 226831_at | 3.36 | 141.02 | 3.35 | 140.25 | 1.00 |
| NM_003410 | ZFX | 207920_x_at | 3.82 | 214.68 | 3.35 | 187.37 | 0.88 |
| NM_022969 | FGFR2 | 203638_s_at | 3.34 | 190.95 | 4.08 | 232.45 | 1.22 |
| AI926697 | Gup1 | 228568_at | 3.37 | 31.46 | 3.34 | 30.70 | 0.99 |
| N30649 | SQSTM1 | 213112_s_at | 6.30 | 175.09 | 3.33 | 92.25 | 0.53 |
| NM_013445 | GAD1 | 206670_s_at | 4.56 | 104.39 | 3.33 | 75.96 | 0.73 |
| AW339310 | DTNA | 227084_at | 3.91 | 190.10 | 3.31 | 160.36 | 0.85 |
| NM_005534 | IFNGR2 | 201642_at | 4.54 | 623.28 | 3.29 | 449.84 | 0.72 |
| NM_000846 | GSTA2 | 203924_at | 5.65 | 291.96 | 3.27 | 169.31 | 0.58 |
| NM_022873 | G1P3 | 204415_at | 5.92 | 2666.37 | 3.27 | 1467.82 | 0.55 |
| BF342851 | D2S448 | 212012_at | 3.59 | 98.45 | 3.27 | 89.15 | 0.91 |
| NM_014398 | LAMP3 | 205569_at | 3.25 | 40.50 | 3.45 | 42.83 | 1.06 |
| AL117653 | MITF | 226066_at | 4.10 | 110.70 | 3.22 | 87.07 | 0.79 |
| AW263086 | KIAA1961 | 228250_at | 3.21 | 56.47 | 4.03 | 70.59 | 1.25 |
| AI734993 | OACT1 | 227379_at | 5.22 | 701.28 | 3.20 | 427.30 | 0.61 |
| NM_006542 | SPHAR | 206272_at | 3.19 | 86.06 | 3.66 | 98.12 | 1.15 |
| AL049699 | RWDD2 | 213555_at | 3.18 | 154.52 | 3.19 | 154.57 | 1.00 |
| U37546 | BIRC3 | 210538_s_at | 3.90 | 120.59 | 3.17 | 98.17 | 0.81 |
| AI133452 | FGG | 226621_at | 3.38 | 377.64 | 3.17 | 354.43 | 0.94 |
| AV700030 | IL6R | 226333_at | 3.14 | 55.50 | 3.73 | 65.78 | 1.19 |
| BF513121 | | 226189_at | 3.13 | 87.02 | 4.32 | 119.55 | 1.38 |
| BE880703 | SGPP1 | 223391_at | 3.83 | 1629.37 | 3.12 | 1320.37 | 0.81 |
| AW444761 | CDKN2B | 236313_at | 3.17 | 45.67 | 3.11 | 44.61 | 0.98 |
| BG231554 | | 243179_at | 3.10 | 50.04 | 4.51 | 72.43 | 1.45 |
| AW006750 | DRE1 | 221986_s_at | 3.78 | 219.28 | 3.09 | 178.93 | 0.82 |
| X83493 | FAS | 215719_x_at | 3.08 | 31.58 | 3.28 | 33.44 | 1.06 |
| AK000004 | FGD3 | 227811_at | 5.70 | 312.89 | 3.08 | 168.55 | 0.54 |
| D42043 | RAFTLIN | 212646_at | 3.08 | 45.61 | 3.60 | 52.97 | 1.17 |
| BF510581 | BTBD11 | 228570_at | 3.08 | 52.05 | 3.30 | 55.79 | 1.07 |
| AU144565 | EPB41L4A | 228256_s_at | 3.99 | 67.18 | 3.07 | 51.46 | 0.77 |
| BE545756 | ADD3 | 201034_at | 4.44 | 330.66 | 3.06 | 226.51 | 0.69 |
| NM_147174 | HS6ST2 | 1552767_a_at | 3.05 | 245.34 | 5.02 | 401.74 | 1.64 |
| BF983406 | HNRPH1 | 213470_s_at | 3.05 | 574.43 | 3.08 | 578.32 | 1.01 |
| NM_000416 | IFNGR1 | 202727_s_at | 3.04 | 906.08 | 3.09 | 921.34 | 1.02 |
| NM_017410 | HOXC13 | 219832_s_at | 5.55 | 1217.29 | 3.04 | 664.86 | 0.55 |
| D28124 | NBL1 | 37005_at | 3.97 | 241.19 | 3.04 | 183.39 | 0.76 |
| NM_014965 | OIP106 | 202080_s_at | 3.30 | 167.17 | 3.03 | 152.45 | 0.92 |
| U84138 | RAD51L1 | 210255_at | 3.12 | 64.11 | 3.01 | 61.69 | 0.97 |
| U78168 | RAPGEF3 | 210051_at | 3.01 | 16.71 | 3.04 | 16.62 | 1.01 |
| AI923675 | MGC19764 | 238430_x_at | 4.51 | 218.01 | 3.00 | 145.32 | 0.67 |
| AL831862 | | 1558142_at | 2.98 | 45.42 | 3.25 | 49.03 | 1.09 |
| W65369 | SETDB2 | 235338_s_at | 2.98 | 56.85 | 3.22 | 61.45 | 1.08 |
| U53823 | OCLN | 209925_at | 3.52 | 394.57 | 2.97 | 333.44 | 0.84 |
| BC001427 | SLC35C1 | 222647_at | 4.70 | 566.64 | 2.95 | 353.12 | 0.63 |
| BE671084 | ARHGAP26 | 205068_s_at | 4.80 | 60.32 | 2.94 | 36.82 | 0.61 |
| AF327722 | NARG1 | 222837_s_at | 2.93 | 110.20 | 3.14 | 117.55 | 1.07 |
| N80935 | | 49111_at | 3.90 | 159.63 | 2.92 | 118.92 | 0.75 |
| BE349147 | CPD | 201941_at | 2.91 | 128.64 | 3.45 | 152.64 | 1.19 |
| AF251053 | BEX2 | 224367_at | 5.29 | 954.18 | 2.90 | 520.98 | 0.55 |
| NM_018418 | SPATA7 | 219583_s_at | 2.90 | 65.43 | 4.42 | 99.44 | 1.53 |
| NM_000632 | ITGAM | 205786_s_at | 2.89 | 102.19 | 3.07 | 108.38 | 1.06 |
| NM_021980 | OPTN | 202074_s_at | 3.95 | 403.35 | 2.89 | 294.45 | 0.73 |
| NM_152569 | C9orf66 | 1552755_at | 4.88 | 358.49 | 2.89 | 211.05 | 0.59 |
| AB040897 | RANBP10 | 221809_at | 2.88 | 60.91 | 2.98 | 62.86 | 1.04 |
| U73844 | ELF3 | 210827_s_at | 4.89 | 1553.45 | 2.88 | 911.04 | 0.59 |
| AI289311 | JUB | 225806_at | 3.02 | 423.47 | 2.88 | 401.35 | 0.95 |
| NM_003060 | SLC22A5 | 205074_at | 4.96 | 511.28 | 2.88 | 295.07 | 0.58 |
| BE500977 | SULF1 | 212354_at | 2.87 | 360.11 | 2.94 | 368.65 | 1.02 |
| NM_001144 | AMFR | 202203_s_at | 2.89 | 58.57 | 2.87 | 57.97 | 0.99 |
| AF307097 | ZNF317 | 1555337_a_at | 3.05 | 57.91 | 2.87 | 54.27 | 0.94 |
| NM_006018 | GPR109B | 205220_at | 3.39 | 33.99 | 2.86 | 28.59 | 0.84 |
| BG109855 | | 213169_at | 2.86 | 43.73 | 3.80 | 58.18 | 1.33 |
| AA128023 | STARD13 | 213103_at | 3.26 | 76.17 | 2.85 | 66.50 | 0.88 |
| AA017721 | | 214046_at | 3.35 | 290.74 | 2.84 | 246.24 | 0.85 |
| NM_003561 | PLA2G10 | 207222_at | 4.57 | 605.12 | 2.83 | 370.32 | 0.62 |
| AK022549 | CDW92 | 224595_at | 2.83 | 1133.49 | 3.02 | 1203.29 | 1.07 |
| AK025862 | LRRC28 | 216450_x_at | 2.89 | 114.24 | 2.82 | 110.84 | 0.98 |
| NM_020639 | RIPK4 | 221215_s_at | 2.82 | 244.86 | 3.50 | 304.12 | 1.24 |
| AY028896 | CARD10 | 210026_s_at | 2.82 | 161.03 | 2.95 | 167.14 | 1.04 |
| NM_006290 | TNFAIP3 | 202644_s_at | 2.82 | 310.68 | 2.84 | 312.79 | 1.01 |
| AI744280 | NBEA | 239010_at | 2.90 | 27.11 | 2.82 | 26.24 | 0.97 |
| AA485908 | INSR | 213792_s_at | 4.88 | 130.76 | 2.82 | 75.14 | 0.58 |
| BE501881 | | 227565_at | 3.22 | 134.99 | 2.82 | 117.63 | 0.87 |
| AI023320 | | 229243_at | 3.18 | 23.57 | 2.81 | 20.72 | 0.88 |
| C15005 | GRPEL2 | 238427_at | 3.30 | 38.73 | 2.81 | 32.96 | 0.85 |
| AA128261 | SAT2 | 225272_at | 3.20 | 291.16 | 2.81 | 254.49 | 0.88 |
| NM_001453 | FOXC1 | 1553613_s_at | 2.93 | 219.31 | 2.81 | 208.93 | 0.96 |
| NM_018333 | PRPF39 | 220553_s_at | 3.03 | 229.07 | 2.80 | 210.95 | 0.92 |
| AK025344 | | 225443_at | 2.80 | 71.28 | 3.01 | 76.60 | 1.07 |
| AI356412 | LYN | 202625_at | 2.80 | 215.02 | 2.99 | 228.85 | 1.07 |
| BF218115 | HIPK2 | 225368_at | 3.67 | 415.92 | 2.79 | 314.85 | 0.76 |
| AL157484 | | 221861_at | 3.60 | 136.58 | 2.79 | 105.25 | 0.77 |
| NM_016044 | FAHD2A | 218504_at | 4.38 | 796.55 | 2.79 | 504.75 | 0.64 |
| BF983948 | SRPRB | 222532_at | 2.78 | 483.56 | 3.15 | 544.60 | 1.13 |
| AI660243 | TMPRSS2 | 226553_at | 4.46 | 823.88 | 2.78 | 505.81 | 0.62 |
| NM_002886 | RAP2B | 214487_s_at | 3.18 | 117.10 | 2.77 | 101.58 | 0.87 |
| NM_001197 | BIK | 205780_at | 2.97 | 539.38 | 2.76 | 502.33 | 0.93 |
| NM_018013 | FLJ10159 | 218974_at | 2.75 | 48.91 | 3.58 | 63.43 | 1.30 |
| AA191573 | SYNJ2 | 212828_at | 5.31 | 294.31 | 2.73 | 150.91 | 0.51 |
| AK026921 | SLC17A5 | 223441_at | 2.73 | 112.31 | 2.85 | 115.81 | 1.05 |
| NM_006895 | HNMT | 204112_s_at | 2.73 | 213.78 | 3.05 | 238.34 | 1.12 |
| AA576961 | PHLDA1 | 217996_at | 3.73 | 1335.09 | 2.72 | 969.32 | 0.73 |
| AK026747 | LOC54103 | 222150_s_at | 4.52 | 151.66 | 2.72 | 90.79 | 0.60 |
| AW511222 | | 241879_at | 3.57 | 362.20 | 2.71 | 275.99 | 0.76 |
| NM_022748 | TENS1 | 217853_at | 3.60 | 948.52 | 2.71 | 711.72 | 0.75 |
| AW028110 | KIAA0500 | 213839_at | 2.70 | 64.17 | 2.92 | 68.29 | 1.08 |
| D87811 | GATA6 | 210002_at | 4.78 | 327.81 | 2.70 | 184.29 | 0.56 |
| U10473 | B4GALT1 | 216627_s_at | 4.42 | 69.07 | 2.70 | 42.00 | 0.61 |
| M25915 | CLU | 208792_s_at | 3.42 | 2463.74 | 2.70 | 1935.88 | 0.79 |
| NM_006705 | GADD45G | 204121_at | 3.85 | 140.57 | 2.69 | 98.43 | 0.70 |
| AI982758 | | 228328_at | 4.44 | 308.45 | 2.69 | 186.11 | 0.61 |
| NM_006399 | BATF | 205965_at | 2.68 | 570.94 | 2.94 | 622.78 | 1.10 |
| AB004064 | TMEFF2 | 224321_at | 3.41 | 124.71 | 2.67 | 97.26 | 0.78 |
| AF070596 | LOC57146 | 213272_s_at | 2.67 | 199.45 | 3.76 | 280.60 | 1.41 |
| NM_002341 | LTB | 207339_s_at | 2.66 | 181.30 | 2.72 | 185.06 | 1.02 |
| NM_014456 | PDCD4 | 202731_at | 2.66 | 1097.30 | 2.81 | 1162.07 | 1.06 |
| BF970340 | LOC339448 | 238010_at | 2.66 | 95.57 | 2.87 | 102.85 | 1.08 |
| NM_003174 | SVIL | 202565_s_at | 2.68 | 773.23 | 2.66 | 767.29 | 0.99 |
| AI758950 | SLC26A7 | 239006_at | 3.24 | 38.62 | 2.65 | 31.46 | 0.82 |
| NM_017420 | SIX4 | 231797_at | 3.41 | 47.47 | 2.64 | 36.62 | 0.77 |
| AB051495 | KIF21A | 226003_at | 4.17 | 3244.69 | 2.64 | 2045.86 | 0.63 |
| AP001743 | ANKRD3 | 234730_s_at | 2.64 | 41.03 | 3.05 | 47.26 | 1.16 |
| AI700506 | RAD52 | 236042_at | 2.64 | 22.22 | 3.01 | 25.28 | 1.14 |
| AI963605 | | 230269_at | 5.22 | 84.87 | 2.63 | 42.86 | 0.50 |
| AW628045 | CXorf23 | 232087_at | 3.45 | 66.01 | 2.63 | 50.07 | 0.76 |
| AV720650 | KIAA0888 | 235048_at | 2.82 | 108.27 | 2.62 | 100.32 | 0.93 |
| AW265065 | | 235079_at | 2.96 | 51.84 | 2.62 | 45.83 | 0.89 |
| AL049385 | KLHL5 | 232297_at | 3.99 | 169.17 | 2.62 | 110.45 | 0.66 |
| NM_007107 | SSR3 | 217790_s_at | 2.61 | 245.65 | 3.05 | 285.83 | 1.17 |
| BE788984 | GALM | 235256_s_at | 2.91 | 84.42 | 2.61 | 75.33 | 0.90 |
| AI357376 | NEDD4L | 212445_s_at | 3.82 | 287.63 | 2.60 | 195.39 | 0.68 |
| AA532655 | FLJ39739 | 229872_s_at | 3.71 | 501.92 | 2.59 | 349.11 | 0.70 |
| NM_020169 | LXN | 218729_at | 3.60 | 11822.71 | 2.59 | 8491.81 | 0.72 |
| BF690020 | EEF1D | 213087_s_at | 4.74 | 117.76 | 2.58 | 63.87 | 0.55 |
| AA133285 | | 230383_x_at | 2.58 | 42.20 | 3.65 | 59.46 | 1.41 |
| AF183569 | ARTS-1 | 209788_s_at | 3.83 | 242.63 | 2.58 | 162.69 | 0.67 |
| BE620832 | CPEB4 | 224831_at | 2.58 | 65.65 | 2.64 | 66.93 | 1.02 |
| AL118571 | C10orf74 | 235016_at | 3.27 | 127.47 | 2.57 | 98.89 | 0.79 |
| AL096776 | RHOU | 223168_at | 2.57 | 220.76 | 3.25 | 277.82 | 1.26 |
| AI829724 | MEF2D | 225641_at | 2.66 | 49.54 | 2.57 | 47.56 | 0.97 |
| BF979668 | C9orf41 | 241781_at | 2.57 | 15.92 | 3.67 | 22.75 | 1.43 |
| AFFX-r2-Bs-phe-5 | | AFFX-r2-Bs-phe- | 2.57 | 81.83 | 2.68 | 84.08 | 1.04 |
| | | 5_at | | | | | |
| BF432550 | MYO1B | 212364_at | 3.66 | 2326.05 | 2.56 | 1621.27 | 0.70 |
| BG491844 | JUN | 201464_x_at | 2.58 | 487.30 | 2.56 | 483.10 | 1.00 |
| NM_003411 | ZFY | 207247_s_at | 2.65 | 70.76 | 2.56 | 68.22 | 0.97 |
| AL136865 | ZNF38 | 223424_s_at | 2.56 | 210.65 | 2.71 | 223.37 | 1.06 |
| NM_016205 | PDGFC | 218718_at | 3.39 | 35.80 | 2.55 | 26.88 | 0.75 |
| U86755 | ADAM17 | 205746_s_at | 2.55 | 103.29 | 2.65 | 107.03 | 1.04 |
| NM_005766 | FARP1 | 201911_s_at | 4.07 | 1092.20 | 2.55 | 680.63 | 0.63 |
| AI344311 | PLDN | 224883_at | 2.60 | 123.10 | 2.54 | 120.42 | 0.98 |
| AL110191 | DSIPI | 208763_s_at | 2.84 | 1914.54 | 2.54 | 1705.15 | 0.90 |
| NM_023039 | ANKRA2 | 218769_s_at | 2.54 | 158.41 | 2.56 | 159.49 | 1.01 |
| AW269686 | RAP2B | 238622_at | 2.54 | 57.07 | 3.60 | 80.69 | 1.42 |
| NM_020995 | HPR | 208470_s_at | 2.54 | 41.48 | 2.71 | 44.30 | 1.07 |
| J03068 | APEH | 201283_s_at | 4.54 | 56.00 | 2.53 | 31.31 | 0.56 |
| BF940761 | | 229427_at | 3.50 | 119.92 | 2.53 | 86.34 | 0.72 |
| NM_005110 | GFPT2 | 205100_at | 2.53 | 42.76 | 2.90 | 49.11 | 1.15 |
| AV688972 | | 239314_at | 2.53 | 48.47 | 2.94 | 56.06 | 1.16 |
| AW973177 | | 236150_at | 2.53 | 70.24 | 2.90 | 79.84 | 1.15 |
| NM_020240 | CDC42SE2 | 1552613_s_at | 3.01 | 162.93 | 2.52 | 135.73 | 0.84 |
| AI400463 | LOC255326 | 229428_at | 2.51 | 56.43 | 3.35 | 74.90 | 1.33 |
| NM_030751 | TCF8 | 208078_s_at | 4.59 | 114.57 | 2.51 | 61.84 | 0.55 |
| AI139252 | STAT3 | 225289_at | 2.81 | 195.93 | 2.50 | 173.85 | 0.89 |
| BE222389 | PCDHB4 | 240317_at | 2.50 | 96.31 | 2.71 | 103.68 | 1.09 |
| AA769615 | MGC2610 | 230434_at | 3.35 | 753.00 | 2.49 | 556.99 | 0.74 |
| BG252842 | C6orf62 | 213875_x_at | 2.58 | 558.60 | 2.49 | 536.27 | 0.97 |
| AB004903 | SOCS2 | 203372_s_at | 2.53 | 109.80 | 2.48 | 107.94 | 0.98 |
| BG054987 | RHPN2 | 227196_at | 3.09 | 915.59 | 2.48 | 735.43 | 0.80 |
| AK000002 | ABCC10 | 213485_s_at | 2.48 | 219.84 | 3.01 | 267.10 | 1.21 |
| AA094434 | SLC8A1 | 241752_at | 2.48 | 32.14 | 3.82 | 49.18 | 1.54 |
| AL563572 | MtFMT | 235689_at | 2.48 | 150.09 | 2.55 | 154.84 | 1.03 |
| BF221852 | LPP | 202822_at | 2.48 | 228.89 | 2.83 | 260.44 | 1.14 |
| D85181 | SC5DL | 211423_s_at | 3.59 | 1312.76 | 2.47 | 901.58 | 0.69 |
| NM_000310 | PPT1 | 200975_at | 2.89 | 2584.14 | 2.47 | 2202.42 | 0.86 |
| NM_024491 | Cep70 | 219036_at | 2.72 | 131.71 | 2.47 | 118.92 | 0.91 |
| NM_016289 | CAB39 | 217873_at | 2.51 | 1808.99 | 2.47 | 1771.89 | 0.98 |
| BF432276 | | 242300_at | 2.45 | 66.68 | 2.91 | 79.04 | 1.19 |
| BE893995 | | 234983_at | 2.72 | 1368.09 | 2.45 | 1225.14 | 0.90 |
| NM_016531 | KLF3 | 219657_s_at | 2.49 | 80.60 | 2.45 | 78.90 | 0.98 |
| NM_014938 | MONDOA | 202519_at | 3.10 | 132.86 | 2.44 | 104.04 | 0.79 |
| AB029031 | TBC1D1 | 212350_at | 2.76 | 138.39 | 2.44 | 121.45 | 0.88 |
| NM_017412 | FZD3 | 219683_at | 2.42 | 219.49 | 2.78 | 251.81 | 1.15 |
| AL136924 | RIN2 | 209684_at | 2.42 | 72.94 | 3.14 | 94.20 | 1.30 |
| AI652868 | | 225811_at | 2.42 | 54.00 | 3.79 | 84.11 | 1.57 |
| AW117765 | PEX13 | 1556009_at | 3.96 | 493.45 | 2.41 | 299.46 | 0.61 |
| AI810572 | PGPEP1 | 237202_at | 2.56 | 70.89 | 2.41 | 65.77 | 0.94 |
| AW157094 | ID4 | 209291_at | 2.41 | 159.87 | 2.47 | 163.03 | 1.03 |
| AI627850 | | 230446_at | 2.41 | 92.83 | 2.67 | 103.26 | 1.11 |
| AV726376 | | 238436_s_at | 2.41 | 18.14 | 2.71 | 20.36 | 1.13 |
| NM_001038 | SCNN1A | 203453_at | 3.33 | 1077.74 | 2.40 | 766.55 | 0.72 |
| AI494047 | | 242898_at | 2.48 | 40.19 | 2.40 | 38.32 | 0.97 |
| NM_005059 | RLN2 | 214519_s_at | 2.39 | 509.31 | 3.19 | 678.67 | 1.33 |
| NM_018360 | CXorf15 | 219969_at | 2.39 | 35.84 | 3.86 | 57.92 | 1.61 |
| BE910071 | CXorf39 | 242781_at | 2.39 | 58.08 | 3.47 | 84.19 | 1.46 |
| AI141556 | BAIAP1 | 225474_at | 2.49 | 118.05 | 2.38 | 112.27 | 0.96 |
| NM_018638 | ETNK1 | 219017_at | 2.38 | 145.88 | 3.84 | 235.19 | 1.61 |
| AB020663 | DMXL2 | 212820_at | 2.38 | 153.06 | 2.89 | 184.77 | 1.21 |
| NM_030952 | SNARK | 220987_s_at | 2.57 | 136.78 | 2.38 | 126.26 | 0.93 |
| NM_014298 | QPRT | 204044_at | 2.93 | 781.72 | 2.37 | 629.21 | 0.81 |
| AA042983 | | 227755_at | 2.37 | 84.14 | 3.14 | 111.18 | 1.33 |
| NM_013352 | SART2 | 218854_at | 2.37 | 60.04 | 2.94 | 74.25 | 1.24 |
| BF115054 | DKFZP564D166 | 224952_at | 2.42 | 238.96 | 2.37 | 232.67 | 0.98 |
| NM_016315 | GULP1 | 204237_at | 2.99 | 693.55 | 2.36 | 545.83 | 0.79 |
| BF674052 | VMP1 | 224917_at | 4.34 | 2881.95 | 2.36 | 1563.97 | 0.54 |
| NM_002801 | PSMB10 | 202659_at | 4.27 | 1030.67 | 2.36 | 568.29 | 0.55 |
| AW628987 | | 225522_at | 3.21 | 266.24 | 2.36 | 194.86 | 0.73 |
| U55936 | SNAP23 | 209131_s_at | 2.35 | 63.81 | 4.13 | 111.25 | 1.75 |
| NM_003810 | TNFSF10 | 202688_at | 2.35 | 271.95 | 2.44 | 280.42 | 1.04 |
| AI492376 | | 231195_at | 2.44 | 26.48 | 2.35 | 25.46 | 0.97 |
| BG434174 | SBLF | 213413_at | 2.35 | 16.59 | 2.69 | 18.91 | 1.14 |
| NM_014840 | ARK5 | 204589_at | 2.35 | 600.19 | 2.70 | 688.00 | 1.15 |
| AI823592 | KIAA0423 | 213304_at | 2.84 | 710.46 | 2.35 | 587.69 | 0.83 |
| AW973842 | C10orf46 | 227257_s_at | 2.86 | 116.82 | 2.34 | 95.21 | 0.82 |
| H17038 | | 226612_at | 2.34 | 140.16 | 3.89 | 231.77 | 1.66 |
| AW043602 | KIAA1946 | 227370_at | 2.33 | 448.54 | 2.95 | 565.19 | 1.26 |
| NM_005724 | TM4SF8 | 200973_s_at | 2.70 | 487.38 | 2.33 | 417.53 | 0.86 |
| AB037730 | KLHL13 | 227875_at | 2.76 | 132.63 | 2.32 | 111.23 | 0.84 |
| BG339050 | | 225155_at | 2.33 | 4861.54 | 2.32 | 4828.85 | 1.00 |
| NM_022071 | SH2D4A | 219749_at | 2.40 | 157.60 | 2.32 | 152.42 | 0.97 |
| AW169333 | CBWD1 | 229804_x_at | 2.39 | 208.49 | 2.32 | 201.58 | 0.97 |
| NM_001621 | AHR | 202820_at | 2.92 | 475.93 | 2.32 | 376.43 | 0.79 |
| NM_000821 | GGCX | 205351_at | 2.47 | 100.74 | 2.32 | 94.15 | 0.94 |
| AW196959 | C14orf150 | 235025_at | 2.93 | 245.06 | 2.32 | 193.14 | 0.79 |
| AK026966 | AK3 | 225342_at | 2.32 | 94.53 | 2.57 | 105.14 | 1.11 |
| AB046783 | ALS2 | 226291_at | 2.31 | 72.27 | 2.32 | 72.40 | 1.01 |
| AI394529 | PRKAG1 | 227527_at | 2.32 | 100.11 | 2.31 | 99.00 | 0.99 |
| AB023179 | KIAA0962 | 212911_at | 2.76 | 57.87 | 2.30 | 47.84 | 0.83 |
| NM_006736 | DNAJB2 | 202500_at | 2.63 | 58.90 | 2.30 | 51.69 | 0.88 |
| AW340595 | | 227384_s_at | 2.37 | 17.36 | 2.29 | 16.75 | 0.97 |
| AI700633 | | 212812_at | 2.92 | 2650.99 | 2.29 | 2085.02 | 0.79 |
| AW006441 | MGC52110 | 226039_at | 2.33 | 130.35 | 2.29 | 127.26 | 0.98 |
| AA724565 | MGC34732 | 237160_at | 2.28 | 164.45 | 2.89 | 207.96 | 1.26 |
| AW069729 | ACPL2 | 226925_at | 3.74 | 198.56 | 2.28 | 120.75 | 0.61 |
| N79004 | SIX1 | 228347_at | 4.55 | 814.67 | 2.28 | 402.32 | 0.50 |
| NM_020190 | OLFML3 | 218162_at | 2.28 | 86.09 | 2.40 | 90.78 | 1.05 |
| NM_001206 | KLF9 | 203543_s_at | 2.35 | 86.60 | 2.28 | 83.26 | 0.97 |
| AI279819 | LOC400451 | 221880_s_at | 4.10 | 941.69 | 2.28 | 521.07 | 0.56 |
| AW592684 | LIFR | 227771_at | 3.60 | 27.29 | 2.28 | 17.15 | 0.63 |
| X96588 | RYK | 216976_s_at | 2.27 | 329.39 | 2.76 | 398.01 | 1.21 |
| AL136710 | ANK3 | 209442_x_at | 2.27 | 304.95 | 2.32 | 309.69 | 1.02 |
| U77914 | JAG1 | 216268_s_at | 2.29 | 217.63 | 2.27 | 215.99 | 0.99 |
| AW206602 | ZSCAN2 | 231188_at | 3.29 | 116.44 | 2.27 | 80.11 | 0.69 |
| AW779917 | | 230003_at | 3.23 | 140.21 | 2.27 | 98.01 | 0.70 |
| AL565362 | SLC2A13 | 227176_at | 2.43 | 77.10 | 2.27 | 71.58 | 0.93 |
| AF285167 | ABCA1 | 203505_at | 2.26 | 229.07 | 2.43 | 245.26 | 1.07 |
| AI984074 | RPL7 | 239493_at | 2.49 | 20.26 | 2.26 | 18.33 | 0.91 |
| AU154321 | KPNA6 | 212101_at | 2.26 | 91.01 | 2.53 | 101.27 | 1.12 |
| NM_018133 | FLJ10546 | 218733_at | 2.31 | 167.84 | 2.26 | 163.56 | 0.98 |
| AF041209 | MID1 | 210694_s_at | 2.26 | 25.14 | 2.57 | 28.46 | 1.14 |
| NM_002925 | RGS10 | 204319_s_at | 3.05 | 915.59 | 2.26 | 679.43 | 0.74 |
| BF968057 | IRF2BP2 | 224571_at | 2.40 | 587.78 | 2.25 | 550.05 | 0.94 |
| AI139990 | | 231022_at | 2.74 | 129.26 | 2.25 | 105.66 | 0.82 |
| W73272 | PDE8A | 212522_at | 4.47 | 1611.41 | 2.25 | 808.15 | 0.50 |
| AA812993 | LOC120376 | 228338_at | 2.25 | 76.76 | 2.81 | 95.98 | 1.25 |
| BC005352 | TNFAIP8 | 210260_s_at | 2.33 | 107.84 | 2.25 | 103.79 | 0.97 |
| AF230929 | ANXA9 | 210085_s_at | 2.24 | 1089.22 | 2.27 | 1097.48 | 1.01 |
| BE675337 | GSN | 214040_s_at | 2.24 | 121.96 | 2.79 | 150.96 | 1.24 |
| AF339824 | HS6ST3 | 232276_at | 2.71 | 65.50 | 2.24 | 53.88 | 0.83 |
| AI799018 | EPHA4 | 227449_at | 2.24 | 57.87 | 2.91 | 74.96 | 1.30 |
| NM_004703 | RABEP1 | 203223_at | 3.03 | 338.02 | 2.24 | 249.75 | 0.74 |
| NM_003916 | AP1S2 | 203300_x_at | 2.69 | 46.41 | 2.23 | 38.20 | 0.83 |
| AF131850 | EI24 | 216396_s_at | 2.23 | 326.91 | 2.63 | 385.74 | 1.18 |
| BF109906 | | 239153_at | 2.81 | 236.51 | 2.23 | 186.98 | 0.79 |
| NM_004114 | FGF13 | 205110_s_at | 2.93 | 542.42 | 2.23 | 413.32 | 0.76 |
| NM_015400 | SMAD3 | 218284_at | 4.25 | 495.99 | 2.23 | 259.65 | 0.53 |
| BF435123 | MSI2 | 225237_s_at | 2.28 | 286.52 | 2.23 | 278.13 | 0.98 |
| BE889301 | | 244007_at | 2.54 | 19.88 | 2.23 | 17.34 | 0.88 |
| NM_024610 | HSPBAP1 | 219284_at | 2.23 | 87.46 | 2.23 | 87.47 | 1.00 |
| BG289443 | | 244561_at | 3.27 | 86.30 | 2.22 | 58.31 | 0.68 |
| AB028869 | BIRC5 | 210334_x_at | 2.67 | 161.06 | 2.22 | 131.65 | 0.83 |
| BG230586 | SLC7A6 | 203578_s_at | 2.56 | 60.43 | 2.22 | 52.05 | 0.87 |
| AI631833 | | 227396_at | 2.59 | 369.66 | 2.22 | 315.46 | 0.86 |
| AI554300 | SERPINB1 | 213572_s_at | 4.05 | 474.94 | 2.22 | 259.15 | 0.55 |
| AI827990 | SLC25A16 | 214140_at | 2.94 | 38.56 | 2.22 | 28.94 | 0.75 |
| AF115512 | DNAJB9 | 1554462_a_at | 2.22 | 170.86 | 2.26 | 174.08 | 1.02 |
| NM_014782 | ARMCX2 | 203404_at | 2.21 | 77.36 | 2.84 | 98.80 | 1.28 |
| BC005122 | ARFGAP3 | 202211_at | 2.54 | 203.27 | 2.21 | 175.93 | 0.87 |
| BE973687 | HES1 | 203394_s_at | 2.96 | 984.23 | 2.21 | 731.42 | 0.75 |
| AC007130 | HIBADH | 231955_s_at | 2.61 | 406.74 | 2.21 | 342.56 | 0.85 |
| AI659800 | FLJ38725 | 228937_at | 2.20 | 44.72 | 2.93 | 59.15 | 1.33 |
| BE677761 | MSCP | 221920_s_at | 2.63 | 32.97 | 2.20 | 27.56 | 0.84 |
| BE465380 | ARNT | 231016_s_at | 2.22 | 74.92 | 2.20 | 74.43 | 0.99 |
| AV725364 | MGC35048 | 225511_at | 3.38 | 59.31 | 2.20 | 38.47 | 0.65 |
| BC003637 | DDIT3 | 209383_at | 2.51 | 328.33 | 2.20 | 286.94 | 0.88 |
| NM_002293 | LAMC1 | 200771_at | 4.18 | 869.01 | 2.20 | 455.00 | 0.53 |
| BE897886 | PIGF | 212120_at | 2.20 | 477.85 | 2.20 | 475.86 | 1.00 |
| AL515381 | CORO2A | 227177_at | 2.19 | 65.93 | 3.14 | 92.77 | 1.43 |
| BC000758 | C6orf80 | 209479_at | 2.46 | 364.00 | 2.19 | 322.89 | 0.89 |
| AJ271379 | SPIN3 | 1555882_at | 2.19 | 33.07 | 3.74 | 56.46 | 1.71 |
| AA533109 | LOC401431 | 229094_at | 2.19 | 19.97 | 2.28 | 20.76 | 1.04 |
| AK025446 | CSIG | 212019_at | 2.19 | 31.86 | 2.54 | 36.85 | 1.16 |
| AW450329 | | 226381_at | 2.26 | 196.84 | 2.19 | 190.07 | 0.97 |
| AF225981 | ATP2C1 | 209934_s_at | 2.66 | 32.44 | 2.19 | 26.59 | 0.82 |
| AF277181 | LOC85028 | 223774_at | 2.24 | 105.83 | 2.19 | 103.16 | 0.98 |
| AK074366 | ZNF621 | 1558620_at | 2.19 | 40.94 | 2.90 | 54.13 | 1.33 |
| T87542 | WDFY2 | 227490_at | 3.12 | 80.98 | 2.19 | 56.85 | 0.70 |
| AU154740 | STAF65(gamma) | 201836_s_at | 2.19 | 34.87 | 2.36 | 37.70 | 1.08 |
| AL096842 | MTUS1 | 212096_s_at | 2.47 | 665.60 | 2.18 | 585.71 | 0.88 |
| AA456973 | PC4 | 221727_at | 2.18 | 77.92 | 2.64 | 93.86 | 1.21 |
| BC000185 | CPT1A | 210688_s_at | 2.18 | 119.09 | 2.75 | 149.75 | 1.26 |
| AL573722 | FLJ90024 | 226239_at | 2.30 | 95.20 | 2.18 | 89.69 | 0.95 |
| NM_000332 | ATXN1 | 203232_s_at | 2.18 | 228.34 | 2.70 | 281.10 | 1.24 |
| NM_004289 | NFE2L3 | 204702_s_at | 2.49 | 77.59 | 2.18 | 67.53 | 0.87 |
| BE742268 | SORT1 | 212797_at | 2.59 | 192.09 | 2.18 | 160.59 | 0.84 |
| AW271788 | NDUFB2 | 226391_at | 2.32 | 29.63 | 2.18 | 27.56 | 0.94 |
| AK001291 | NCKAP1 | 217465_at | 2.18 | 132.65 | 2.25 | 136.30 | 1.03 |
| NM_015516 | TSK | 218245_at | 2.83 | 421.10 | 2.17 | 322.17 | 0.77 |
| AB017493 | KLF6 | 208961_s_at | 2.17 | 54.62 | 2.53 | 63.42 | 1.17 |
| NM_019094 | NUDT4 | 206302_s_at | 2.34 | 623.08 | 2.17 | 580.18 | 0.93 |
| AI954660 | C17orf27 | 225931_s_at | 2.27 | 60.55 | 2.17 | 57.70 | 0.96 |
| AK000445 | HOXC9 | 231936_at | 2.45 | 61.55 | 2.17 | 54.04 | 0.89 |
| AA551142 | PHACTR2 | 204048_s_at | 2.85 | 748.78 | 2.17 | 566.64 | 0.76 |
| AI819238 | ID2 | 213931_at | 2.64 | 24.73 | 2.17 | 20.17 | 0.82 |
| BC040952 | PIK3C2A | 1569022_a_at | 2.56 | 68.80 | 2.16 | 58.26 | 0.85 |
| AB040875 | SLC7A11 | 209921_at | 2.47 | 107.04 | 2.16 | 93.19 | 0.87 |
| AW242916 | IL6ST | 212196_at | 2.16 | 473.64 | 2.19 | 482.40 | 1.02 |
| AA100250 | DHX57 | 213420_at | 2.15 | 73.88 | 2.19 | 74.91 | 1.02 |
| AI554514 | | 229796_at | 2.53 | 569.56 | 2.15 | 480.93 | 0.85 |
| AV753204 | MAP3K9 | 213927_at | 2.40 | 74.22 | 2.15 | 66.51 | 0.90 |
| BF000047 | | 235736_at | 2.15 | 60.05 | 2.75 | 76.95 | 1.28 |
| NM_002178 | IGFBP6 | 203851_at | 2.60 | 211.46 | 2.14 | 173.63 | 0.83 |
| AW137982 | HOXA3 | 235521_at | 2.14 | 26.88 | 2.39 | 29.94 | 1.11 |
| AW006123 | FBXO32 | 225803_at | 2.14 | 148.96 | 2.26 | 156.52 | 1.05 |
| BF589251 | | 227776_at | 2.14 | 42.69 | 2.71 | 53.91 | 1.27 |
| AI458439 | | 231929_at | 2.63 | 132.82 | 2.14 | 107.61 | 0.81 |
| AF029750 | TAPBP | 208829_at | 4.00 | 1486.80 | 2.14 | 791.19 | 0.53 |
| NM_020130 | C8orf4 | 218541_s_at | 2.85 | 40.30 | 2.13 | 30.03 | 0.75 |
| NM_022459 | XPO4 | 218479_s_at | 2.89 | 42.90 | 2.13 | 31.14 | 0.74 |
| AA044835 | SLC35F5 | 225872_at | 2.17 | 1064.66 | 2.13 | 1038.98 | 0.98 |
| BF574430 | | 235059_at | 2.13 | 293.65 | 2.13 | 292.33 | 1.00 |
| AW418666 | HINT3 | 226537_at | 2.12 | 111.52 | 2.38 | 125.09 | 1.12 |
| AF151046 | C3orf19 | 223787_s_at | 2.12 | 80.75 | 2.63 | 99.88 | 1.24 |
| NM_012382 | OSRF | 219421_at | 2.31 | 58.43 | 2.11 | 52.97 | 0.91 |
| U75667 | ARG2 | 203946_s_at | 2.11 | 15.09 | 2.18 | 15.34 | 1.03 |
| AF087847 | GABARAPL1 | 208869_s_at | 3.71 | 50.82 | 2.11 | 28.84 | 0.57 |
| NM_019058 | DDIT4 | 202887_s_at | 2.32 | 769.51 | 2.11 | 702.40 | 0.91 |
| AF314544 | TBL1XR1 | 222634_s_at | 2.51 | 122.82 | 2.11 | 103.53 | 0.84 |
| NM_004734 | DCAMKL1 | 205399_at | 2.11 | 65.08 | 2.33 | 71.58 | 1.10 |
| BC019922 | ZNF252 | 1558722_at | 2.17 | 138.44 | 2.11 | 133.91 | 0.97 |
| NM_138995 | MYO3B | 1552578_a_at | 3.34 | 77.32 | 2.11 | 48.65 | 0.63 |
| BC021215 | FLJ11193 | 1552660_a_at | 2.11 | 156.54 | 2.96 | 219.17 | 1.41 |
| AF275800 | MGC5306 | 222728_s_at | 2.10 | 346.15 | 2.30 | 376.61 | 1.09 |
| NM_004363 | CEACAM5 | 201884_at | 3.85 | 95.32 | 2.10 | 51.85 | 0.55 |
| AI990326 | MPHOSPH9 | 221965_at | 2.10 | 14.01 | 2.65 | 17.56 | 1.26 |
| AFFX-M27830_M | | AFFX- | 2.82 | 1856.84 | 2.10 | 1377.96 | 0.74 |
| | | M27830_M_at | | | | | |
| AA196245 | EXT2 | 202012_s_at | 2.15 | 150.26 | 2.10 | 145.60 | 0.97 |
| NM_019061 | PIP3AP | 220953_s_at | 2.09 | 37.79 | 2.68 | 47.78 | 1.28 |
| AB014540 | SWAP70 | 209307_at | 2.23 | 49.60 | 2.09 | 46.26 | 0.94 |
| NM_004578 | RAB4A | 203582_s_at | 2.13 | 778.46 | 2.09 | 766.06 | 0.98 |
| AY007243 | REG4 | 223447_at | 2.09 | 228.34 | 2.17 | 236.02 | 1.04 |
| AK096921 | | 1558105_a_at | 2.79 | 203.69 | 2.09 | 151.59 | 0.75 |
| AA496034 | LOC55971 | 227372_s_at | 2.09 | 252.98 | 2.12 | 256.38 | 1.02 |
| AK056658 | FLJ32096 | 1555870_at | 2.98 | 22.02 | 2.09 | 15.34 | 0.70 |
| AA121673 | ZNF281 | 228785_at | 2.28 | 851.05 | 2.08 | 777.96 | 0.91 |
| BF056095 | TMEM42 | 226361_at | 2.47 | 124.49 | 2.08 | 104.19 | 0.84 |
| AL136855 | DKFZp434K2435 | 223594_at | 2.08 | 111.84 | 2.40 | 129.34 | 1.16 |
| NM_007236 | CHP | 207993_s_at | 2.32 | 73.89 | 2.08 | 65.93 | 0.90 |
| AI432196 | NR3C1 | 201865_x_at | 2.08 | 406.15 | 2.22 | 433.24 | 1.07 |
| NM_003670 | BHLHB2 | 201170_s_at | 3.46 | 329.44 | 2.07 | 196.56 | 0.60 |
| AY038927 | DUSP16 | 1555399_a_at | 2.30 | 28.67 | 2.07 | 25.78 | 0.90 |
| AA025858 | CRTAP | 227138_at | 2.07 | 28.73 | 2.24 | 30.55 | 1.08 |
| AI439752 | SMAD5 | 235451_at | 2.30 | 155.69 | 2.07 | 139.44 | 0.90 |
| AV718192 | TRIO | 209012_at | 2.07 | 38.34 | 2.13 | 39.37 | 1.03 |
| BC002427 | CASP2 | 209811_at | 3.72 | 48.83 | 2.06 | 26.75 | 0.56 |
| NM_002393 | MDM4 | 205655_at | 2.06 | 48.40 | 3.09 | 72.72 | 1.50 |
| AW612407 | | 230098_at | 2.32 | 18.38 | 2.06 | 16.24 | 0.89 |
| NM_005980 | S100P | 204351_at | 2.33 | 1213.97 | 2.06 | 1068.60 | 0.88 |
| NM_000375 | UROS | 203031_s_at | 2.13 | 162.12 | 2.06 | 155.96 | 0.97 |
| X16135 | HNRPL | 35201_at | 2.16 | 513.42 | 2.06 | 485.74 | 0.95 |
| BF699855 | GALNT7 | 222587_s_at | 2.51 | 270.16 | 2.05 | 218.08 | 0.82 |
| BE378670 | MGC9850 | 224857_s_at | 2.58 | 921.93 | 2.05 | 731.54 | 0.80 |
| NM_004414 | DSCR1 | 208370_s_at | 3.64 | 244.75 | 2.05 | 137.53 | 0.56 |
| BC005127 | ADFP | 209122_at | 2.05 | 465.12 | 2.12 | 479.62 | 1.04 |
| N25562 | PTK9 | 214008_at | 2.05 | 45.29 | 2.13 | 46.77 | 1.04 |
| AL136139 | NEDD9 | 202149_at | 2.24 | 1198.86 | 2.05 | 1094.66 | 0.92 |
| NM_018222 | PARVA | 217890_s_at | 2.17 | 160.38 | 2.05 | 150.35 | 0.94 |
| BM968434 | ZNF286 | 1557684_at | 2.29 | 59.17 | 2.05 | 52.67 | 0.89 |
| AI611074 | CDKL5 | 227004_at | 3.20 | 126.90 | 2.05 | 80.97 | 0.64 |
| AA541479 | MAP3K1 | 225927_at | 2.05 | 640.64 | 2.06 | 640.12 | 1.00 |
| NM_000456 | SUOX | 1553030_a_at | 2.05 | 58.46 | 3.53 | 100.04 | 1.73 |
| NM_007173 | PRSS23 | 202458_at | 2.05 | 2332.41 | 2.22 | 2514.53 | 1.08 |
| NM_004090 | DUSP3 | 201538_s_at | 2.94 | 153.75 | 2.05 | 107.27 | 0.70 |
| BF972755 | VHL | 1559227_s_at | 2.05 | 51.65 | 2.67 | 67.60 | 1.31 |
| AJ005866 | SLC35D2 | 213083_at | 2.73 | 256.38 | 2.04 | 191.08 | 0.75 |
| AB002344 | JMJD3 | 41386_i_at | 2.48 | 49.96 | 2.04 | 40.94 | 0.82 |
| AW361702 | PSMD7 | 238738_at | 2.04 | 45.42 | 2.33 | 51.59 | 1.14 |
| AL137370 | DKFZp434H2226 | 232893_at | 2.04 | 17.21 | 2.17 | 18.33 | 1.06 |
| NM_006291 | TNFAIP2 | 202510_s_at | 2.41 | 135.69 | 2.04 | 115.14 | 0.85 |
| BF038869 | | 235322_at | 2.13 | 41.98 | 2.04 | 39.87 | 0.95 |
| BG292233 | INSIG1 | 201626_at | 2.04 | 776.46 | 2.33 | 885.79 | 1.15 |
| AP001745 | PRDM15 | 225539_at | 2.04 | 78.14 | 3.40 | 130.79 | 1.67 |
| AU145950 | TGFB2 | 228121_at | 2.04 | 41.53 | 2.66 | 54.00 | 1.31 |
| NM_003901 | SGPL1 | 208381_s_at | 2.14 | 38.45 | 2.03 | 36.40 | 0.95 |
| AW467077 | LOC284408 | 235779_at | 2.03 | 110.63 | 2.16 | 117.06 | 1.06 |
| AI039874 | NQO1 | 201467_s_at | 2.56 | 6006.97 | 2.03 | 4706.32 | 0.80 |
| NM_020327 | ACVR1B | 208223_s_at | 2.81 | 32.53 | 2.03 | 23.29 | 0.72 |
| AW268719 | | 236798_at | 2.03 | 157.04 | 2.83 | 218.47 | 1.40 |
| BC046206 | ZNF26 | 1555325_s_at | 2.03 | 64.93 | 3.67 | 116.69 | 1.81 |
| AI184512 | CTMP | 229253_at | 2.03 | 24.87 | 2.24 | 27.30 | 1.10 |
| BG236163 | KIAA1219 | 221738_at | 2.03 | 38.37 | 2.95 | 55.53 | 1.45 |
| AK023014 | ARHGAP5 | 233849_s_at | 2.03 | 102.71 | 2.13 | 107.30 | 1.05 |
| BF197707 | FLJ30656 | 212529_at | 2.09 | 201.93 | 2.03 | 195.23 | 0.97 |
| J02923 | LCP1 | 208885_at | 2.25 | 189.70 | 2.03 | 169.88 | 0.90 |
| AK022014 | AKAP13 | 222024_s_at | 2.03 | 20.93 | 2.51 | 25.67 | 1.24 |
| N25732 | FOXO3A | 204131_s_at | 2.53 | 1325.26 | 2.02 | 1053.09 | 0.80 |
| NM_022470 | WIG1 | 219628_at | 2.30 | 106.38 | 2.02 | 93.40 | 0.88 |
| NM_019044 | FLJ10996 | 219774_at | 2.02 | 52.41 | 2.25 | 58.25 | 1.11 |
| NM_018357 | FLJ11196 | 218651_s_at | 2.24 | 26.44 | 2.02 | 23.71 | 0.90 |
| NM_013332 | HIG2 | 218507_at | 2.19 | 1320.58 | 2.02 | 1208.52 | 0.92 |
| AW959427 | DNCL2B | 238116_at | 2.02 | 23.80 | 2.69 | 31.70 | 1.34 |
| AL121883 | ARMCX3 | 222444_at | 2.01 | 552.98 | 2.84 | 780.21 | 1.41 |
| BG390493 | STYX | 244030_at | 2.01 | 238.65 | 2.46 | 290.46 | 1.22 |
| L78132 | LGALS8 | 208935_s_at | 2.79 | 300.96 | 2.01 | 213.68 | 0.72 |
| NM_002194 | INPP1 | 202794_at | 2.01 | 30.38 | 2.09 | 31.59 | 1.04 |
| BE965029 | MICAL2 | 212473_s_at | 2.01 | 223.12 | 2.23 | 248.15 | 1.11 |
| BF444943 | USP24 | 212381_at | 2.01 | 64.87 | 2.16 | 69.36 | 1.07 |
| NM_005952 | MT1X | 208581_x_at | 3.10 | 2649.72 | 2.01 | 1719.45 | 0.65 |
| AA337048 | TEAD2 | 238323_at | 2.81 | 124.69 | 2.01 | 87.88 | 0.72 |
| AA001423 | AMIGO | 226718_at | 2.40 | 108.27 | 2.01 | 89.33 | 0.84 |
| NM_004129 | GUCY1B2 | 220506_at | 2.63 | 31.31 | 2.01 | 23.77 | 0.76 |
| AI911972 | | 230780_at | 3.89 | 232.52 | 2.00 | 119.32 | 0.52 |
| AL047052 | LOC23117 | 235060_at | 2.00 | 27.38 | 3.66 | 49.37 | 1.83 |
| AI769569 | MAML2 | 235457_at | 3.92 | 32.21 | 2.00 | 16.47 | 0.51 |
| J04152 | TACSTD2 | 202286_s_at | 2.17 | 10226.30 | 2.00 | 9388.66 | 0.92 |
| CA442342 | | 1556097_at | 3.11 | 30.60 | 2.00 | 19.70 | 0.64 |
| BC000576 | QDPR | 209123_at | 2.02 | 244.71 | 2.00 | 242.32 | 0.99 |
| AI567426 | TLE3 | 212769_at | 2.72 | 98.78 | 2.00 | 71.80 | 0.73 |
| BE504242 | LOC158402 | 236769_at | 2.00 | 12.59 | 3.78 | 23.72 | 1.89 |

TABLE 4
Genes showing strong preferential induction by the RAR agonist relative to the antagonist.

| | | | Maximal effect of | | Maximal effect of | | Relative |
|---|---|---|---|---|---|---|---|
| | | | agonist | | antagonist | | maximal |
| | | Affymetrix probe | Fold | Raw | Fold | Raw | induction |
| Genbank ID | Gene name | ID | induction | signal | induction | signal | (antag/agon) |
| CYP26B1 | NM_019885 | 219825_at | 179.84 | 1122.80 | 2.41 | 14.96 | 0.01 |
| TRIM31 | NM_007028 | 208170_s_at | 35.70 | 280.19 | 1.89 | 14.81 | 0.05 |
| APXL2 | AA588854 | 239435_x_at | 63.13 | 2943.72 | 4.40 | 204.15 | 0.07 |
| CYP26A1 | NM_000783 | 206424_at | 220.23 | 2729.21 | 23.46 | 289.57 | 0.11 |
| HOXA3 | T89711 | 230080_at | 10.38 | 219.24 | 1.11 | 23.33 | 0.11 |
| SLC1A1 | AW235061 | 213664_at | 8.96 | 50.61 | 1.07 | 6.04 | 0.12 |
| RNASE1 | NM_002933 | 201785_at | 49.39 | 706.93 | 5.99 | 85.44 | 0.12 |
| HPS3 | AI922198 | 227253_at | 21.01 | 212.31 | 2.70 | 27.15 | 0.13 |
| KRT4 | X07695 | 213240_s_at | 9.38 | 279.97 | 1.27 | 37.27 | 0.13 |
| CP | AI684991 | 228143_at | 10.88 | 57.74 | 1.52 | 8.09 | 0.14 |
| | CA448125 | 1557050_at | 8.90 | 128.10 | 1.37 | 19.60 | 0.15 |
| IGFBP3 | BF340228 | 212143_s_at | 16.12 | 296.21 | 2.49 | 45.62 | 0.15 |
| HSXIAPAF1 | AA142842 | 228617_at | 17.54 | 396.67 | 2.76 | 62.15 | 0.16 |
| GBP3 | AL136680 | 223434_at | 7.32 | 169.25 | 1.18 | 27.20 | 0.16 |
| TRIM31 | X81006 | 215444_s_at | 12.16 | 235.45 | 2.07 | 39.84 | 0.17 |
| APXL2 | AL138455 | 241935_at | 5.82 | 227.53 | 1.01 | 39.19 | 0.17 |
| CEACAM1 | NM_001712 | 206576_s_at | 16.23 | 236.29 | 2.81 | 40.79 | 0.17 |
| | BF593636 | 228642_at | 130.43 | 964.04 | 23.01 | 169.44 | 0.18 |
| CP | AL556703 | 1558034_s_at | 44.25 | 324.77 | 7.85 | 57.39 | 0.18 |
| OAS2 | NM_016817 | 204972_at | 15.91 | 262.84 | 2.84 | 46.29 | 0.18 |
| | CA448125 | 1557051_s_at | 23.74 | 671.29 | 4.26 | 120.03 | 0.18 |
| GBP1 | BC002666 | 202269_x_at | 5.99 | 47.16 | 1.10 | 8.60 | 0.18 |
| CYP1A1 | NM_000499 | 205749_at | 7.33 | 35.09 | 1.35 | 6.45 | 0.18 |
| LOC134548 | AI744123 | 230238_at | 5.56 | 37.93 | 1.03 | 7.02 | 0.19 |
| IFIT4 | AI075407 | 229450_at | 16.59 | 2294.88 | 3.09 | 426.20 | 0.19 |
| | AK024898 | 227306_at | 11.73 | 166.77 | 2.20 | 31.15 | 0.19 |
| HOXA1 | S79910 | 214639_s_at | 95.33 | 726.74 | 18.35 | 139.31 | 0.19 |
| PCSK2 | NM_002594 | 204870_s_at | 6.32 | 67.97 | 1.24 | 13.27 | 0.20 |
| | AW510657 | 228904_at | 10.13 | 137.42 | 2.05 | 27.49 | 0.20 |
| KDR | NM_002253 | 203934_at | 8.75 | 135.14 | 1.78 | 27.43 | 0.20 |
| HOXA13 | BG289306 | 231786_at | 4.87 | 33.91 | 1.01 | 6.99 | 0.21 |
| LOC145757 | AK056534 | 1558649_at | 4.71 | 25.05 | 1.01 | 5.32 | 0.21 |
| IFI44L | NM_006820 | 204439_at | 7.95 | 36.49 | 1.71 | 7.84 | 0.22 |
| GPX2 | NM_002083 | 202831_at | 38.21 | 1926.26 | 8.34 | 418.80 | 0.22 |
| CP | NM_000096 | 204846_at | 67.09 | 433.38 | 14.65 | 94.27 | 0.22 |
| KIAA1359 | AB037780 | 231941_s_at | 11.07 | 193.70 | 2.46 | 42.96 | 0.22 |
| SELE | NM_000450 | 206211_at | 4.54 | 28.14 | 1.02 | 6.31 | 0.22 |
| PDK4 | AV707102 | 225207_at | 4.76 | 44.76 | 1.08 | 10.14 | 0.23 |
| ChGn | NM_018371 | 219049_at | 13.08 | 216.76 | 3.01 | 50.01 | 0.23 |
| OAS2 | AI651594 | 228607_at | 7.28 | 153.38 | 1.68 | 35.23 | 0.23 |
| CEACAM1 | X16354 | 209498_at | 39.62 | 216.69 | 9.17 | 49.98 | 0.23 |
| PDE5A | AB015656 | 1553175_s_at | 7.55 | 152.03 | 1.76 | 35.26 | 0.23 |
| CEACAM1 | M76742 | 211883_x_at | 4.51 | 146.82 | 1.05 | 34.16 | 0.23 |
| | BF221547 | 227088_at | 26.46 | 253.51 | 6.24 | 59.53 | 0.24 |
| LOC121838 | AI680459 | 232318_s_at | 28.28 | 330.40 | 6.70 | 77.93 | 0.24 |
| FLJ32115 | AI051248 | 227450_at | 173.10 | 2721.33 | 41.18 | 644.97 | 0.24 |
| FLJ11127 | NM_019018 | 219694_at | 22.82 | 199.13 | 5.45 | 47.42 | 0.24 |
| TRIM31 | AF230386 | 210159_s_at | 17.09 | 319.39 | 4.11 | 76.49 | 0.24 |
| LOC129607 | AI742057 | 226702_at | 6.01 | 958.79 | 1.45 | 230.73 | 0.24 |
| TGFBI | NM_000358 | 201506_at | 53.01 | 6739.18 | 12.84 | 1625.97 | 0.24 |
| RSAD2 | AI337069 | 213797_at | 7.54 | 130.76 | 1.83 | 31.61 | 0.24 |
| PDE5A | NM_001083 | 206757_at | 16.48 | 158.21 | 4.03 | 38.56 | 0.24 |
| FLJ10901 | NM_018265 | 219010_at | 4.05 | 328.02 | 1.01 | 81.19 | 0.25 |

What is claimed is:

1. A method for inducing growth arrest of proliferating cells comprising contacting the cells with a retinoic acid receptor (RAR)-modulating compound that induces RARE-independent retinoid-responsive gene expression and that is inefficient in inducing RARE-dependent gene expression.

2. The method according to claim 1, wherein the proliferating cells are neoplastic cells.

3. The method according to claim 1, wherein the proliferating cells are in a human.

4. A method for identifying a RAR-modulating compound that induces growth arrest of proliferating cells and that induces RARE-independent retinoid-responsive gene expression and that is inefficient in inducing RARE-dependent gene expression comprising providing proliferating cells, contacting the cells with a test compound, determining the level of RARE-independent retinoid-responsive gene expression, determining the level of RARE-dependent gene expression and comparing the levels of RARE-independent retinoid-responsive gene expression with the level of RARE-dependent gene expression, wherein a test compound that increases the expression of RARE-independent retinoid-responsive genes relative to cells not treated with the test compound and that produces the highest ratio of the expression of RARE-independent retinoid-responsive genes to the expression of RARE-dependent genes, is determined to be a RAR-modulating compound that is inefficient in inducing RARE-dependent gene expression and induces cell growth arrest.

5. The method according to claim 4, wherein RARE-independent retinoid-responsive gene expression and RARE-dependent gene expression are determined by providing cells transfected with a first gene encoding a first detectable protein operatively linked to a promoter of a RARE-independent retinoid-responsive gene and a second gene encoding a second detectable protein that is different from and separately detectable in the presence of the first detectable protein operatively linked to a promoter of a RARE-dependent gene, measuring the levels of expression of the first and second detectable proteins, and comparing the levels of expression of the first and second detectable proteins.

6. The method according to claim 4, wherein the levels of RARE-independent retinoid-responsive gene expression and RARE-dependent gene expression is further compared to cells treated with a compound known to induce RARE-dependent gene expression.

7. The method according to claim 5, wherein the detectable protein is selected from the group consisting of firefly luciferase, Renilla luciferase, beta-galactosidase, chloramphenicol acetyltransferase, horseradish peroxidase, green fluorescent protein, yellow fluorescent protein, cyan fluorescent protein, fluorescent protein DsRed, alkaline phosphatase and immunologically detectable proteins or peptides.

8. The method according to claim 4, wherein RARE-independent retinoid-responsive gene expression and/or RARE-dependent gene expression is determined by quantitative reverse transcription PCR.

9. The method according to claim 4, wherein RARE-independent retinoid-responsive gene expression and/or RARE-dependent gene expression is determined by microarray analysis.

10. The method according to claim 4, wherein the compounds are derivatives of existing RAR agonists or antagonists.

11. The method according to claim 4, wherein the RARE-independent retinoid-responsive genes are selected from the group consisting of IGF-BP3, EPLIN, FAT10, βIG-H3, RGC32, NR5A2, BDKRB2, EPAS1, LOC283824, CMYA5, A1935586, GK, RUNX2, IL8, SPTBN1, LCN2, PSTPIP2, IBRDC2, GPRC5B, FLJ1017, RAI14, OSMR, FBLN5, SAT, GPCR5A, ABCC4, BTG2, DCDC2, NMES1, DOCK8, C6orf155, SAMD4, CCL20, ASB9, STEAP, BF939996, EIF2S3, RIT1, A1918054, CHI3L2 and COL12A1.

12. The method according to claim 4, wherein the RARE-dependent genes are selected from the group consisting of CYP26A1, CYP26B1, HOXA1, APXL2, RNASE1, CP, CA448125, HPS3, HSXIAPAF1, IFIT4, CEACAM1, IGFBP3, TCRGC2, TRIM31, AK024898, HOXA3, KRT4, SLC1A1, GBP3 and PCSK2.

13. A compound identified by the method of claim 4.

14. The method according to claim 5, wherein the levels of RARE-independent retinoid-responsive gene expression and RARE-dependent gene expression is further compared to cells treated with a compound known to induce RARE-dependent gene expression.

15. The method according to claim 8, wherein the levels of RARE-independent retinoid-responsive gene expression and RARE-dependent gene expression is further compared to cells treated with a compound known to induce RARE-dependent gene expression.

16. The method according to claim 9, wherein the levels of RARE-independent retinoid-responsive gene expression and RARE-dependent gene expression is further compared to cells treated with a compound known to induce RARE-dependent gene expression.


SEQUENCE LISTING (12 sequences)

SEQ ID NO: 1 - DNA, 19 bp, Artificial Sequence
  Description of Artificial Sequence Synthetic primer
ttgcacaaagactgccaag

SEQ ID NO: 2 - DNA, 20 bp, Artificial Sequence
  Description of Artificial Sequence Synthetic primer
aatcagttcaccacaaacag

SEQ ID NO: 3 - DNA, 20 bp, Artificial Sequence
  Description of Artificial Sequence Synthetic primer
catgagggaacggaagcggg

SEQ ID NO: 4 - DNA, 20 bp, Artificial Sequence
  Description of Artificial Sequence Synthetic primer
aacgagcttcttgagatcgt

SEQ ID NO: 5 - DNA, 20 bp, Artificial Sequence
  Description of Artificial Sequence Synthetic primer
aatgcttcctgcctctgtgt

SEQ ID NO: 6 - DNA, 20 bp, Artificial Sequence
  Description of Artificial Sequence Synthetic primer
cgctgtcatatgggttggca

SEQ ID NO: 7 - DNA, 20 bp, Artificial Sequence
  Description of Artificial Sequence Synthetic primer
agaagccgcccattcactgt

SEQ ID NO: 8 - DNA, 20 bp, Artificial Sequence
  Description of Artificial Sequence Synthetic primer
ctgataggtggggacacagt

SEQ ID NO: 9 - DNA, 20 bp, Artificial Sequence
  Description of Artificial Sequence Synthetic primer
tgcgactagcccctgtctat

SEQ ID NO: 10 - DNA, 20 bp, Artificial Sequence
  Description of Artificial Sequence Synthetic primer
gtggtgcattcctcctgtag

SEQ ID NO: 11 - DNA, 18 bp, Artificial Sequence
  Description of Artificial Sequence Synthetic primer
cttcctgggcatggagtc

SEQ ID NO: 12 - DNA, 20 bp, Artificial Sequence
  Description of Artificial Sequence Synthetic primer
tgttggcgtacaggtctttg